(19) United States
(12) Patent Application Publication
(10) Pub. No.: US 2007/0191450 A1
(43) Pub. Date: Aug. 16, 2007

(54) Use of Benzo-Heteroaryl Sulfamide Derivatives for the Treatment of Mania and Bipolar Disorder

(76) Inventor: Virginia L. Smith-Swintosky, Hatfield, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/673,723

(22) Filed: Feb. 12, 2007

Related U.S. Application Data
(60) Provisional application No. 60/773,712, filed on Feb. 15, 2006.

(51) Int. Cl. A61K 31/4184; A61K 31/405; A61K 31/381; A61K 31/343; A61K 31/16

(52) U.S. Cl. 514/397; 514/419; 514/443; 514/469; 514/600

(57) ABSTRACT

The present invention is a method for the treatment of mania and/or bipolar disorder comprising administering to a subject in need thereof a therapeutically effective amount of one or more novel benzo-heteroaryl sulfamide derivatives of formula (I) as herein defined.

### CROSS REFERENCE TO RELATED APPLICATIONS

The application claims the benefit of U.S. Provisional Application 60/773,712, filed on Feb. 15, 2006, which is incorporated by reference herein in it's entirety.

### FIELD OF THE INVENTION

The present invention is directed to the use of benzo-heteroaryl sulfamide derivatives for the treatment of mania and bipolar disorder.

### BACKGROUND OF THE INVENTION

Bipolar disorder is psychiatric disorder characterized by unpredictable swings in mood from mania (or hypomania) to depression. Some patients suffer only from recurrent attacks of mania, which in its pure form is associated with increased psychomotor activity; excessive social extroversion; decreased need for sleep; impulsivity and impairment in judgment; and expansive, grandiose, and sometimes irritable mood. In severe mania, patients may experience delusions and paranoid thinking indistinguishable from schizophrenia. Half of patients with bipolar disorder present with a mixture of psychomotor agitation and activation with dysphoria, anxiety, and irritability. It may be difficult to distinguish mixed mania from agitated depression. In some bipolar patients (bipolar II disorder), the full criteria for mania are lacking, and the requisite recurrent depressions are separated by periods of mild activation and increased energy (hypomania). In cyclothymic disorder, there are numerous hypomanic periods, usually of relatively short duration, alternating with clusters of depressive symptoms that fail, either in severity or duration, to meet the criteria of major depression. The mood fluctuations are chronic and should be present for at least 2 years before the diagnosis is made.

Manic episodes typically emerge over a period of days to weeks, but onset within hours is possible, usually in the early morning hours. An untreated episode of either depression or mania can be as short as several weeks or last as long as 8 to 12 months, and rare patients have an unremitting chronic course. The term rapid cycling is used for patients who have four or more episodes of either depression or mania in a given year. This pattern occurs in 15% of all patients, almost all of whom are women. In some cases, rapid cycling is linked to an underlying thyroid dysfunction and, in others, it is iatrogenically triggered by prolonged antidepressant treatment. Approximately half of patients have sustained difficulties in work performance and psychosocial functioning.

Patients suffering from bipolar disorder typically complain of the following types of symptoms, depending on whether they are in a “manic” or “high” phase versus a “depressed” or “low” phase. In the manic phase symptoms include, but are not limited to (a) increased physical and mental activity and energy (b) heightened mood, exaggerated optimism and self-confidence; (c) excessive irritability, aggressive behavior; (d) decreased need for sleep without experiencing fatigue; (e) grandiose delusions, inflated sense of self-importance; (h) racing speech, racing thoughts, flight of ideas; (i) impulsiveness, poor judgment, distractibility; (j) reckless behavior and In the most severe cases, (k) delusions and hallucinations. In the manic phase symptoms include, but are not limited to (a) prolonged sadness or unexplained crying spells; (b) significant changes in appetite and sleep patterns; (c) irritability, anger, worry, agitation, anxiety; (d) pessimism, indifference; (e) loss of energy, persistent lethargy; (f) feelings of guilt, worthlessness; (g) inability to concentrate, indecisiveness; (h) inability to take pleasure in former interests, social withdrawal; (i) unexplained aches and pains and (j) recurring thoughts of death or suicide.

Bipolar disorder is common, affecting ˜1% of the population in the United States. Onset is typically between 20 and 30 years of age, but many individuals report premorbid symptoms in late childhood or early adolescence. The prevalence is similar for men and women; women are likely to have more depressive and men more manic episodes over a lifetime.

Lithium carbonate is the mainstay of treatment in bipolar disorder, although sodium valproate and olanzapine are equally effective in acute mania, as is lamotrigine in the depressed phase. The response rate to lithium carbonate is 70 to 80% in acute mania, with beneficial effects appearing in 1 to 2 weeks. Lithium also has a prophylactic effect in prevention of recurrent mania and, to a lesser extent, in the prevention of recurrent depression. Serious side effects from lithium administration are rare, but minor complaints such as gastrointestinal discomfort, nausea, diarrhea, polyuria, weight gain, skin eruptions, alopecia, and edema are common.

In the treatment of acute mania, lithium is initiated at 300 mg bid or tid, and the dose is then increased by 300 mg every 2 to 3 days to achieve blood levels of 0.8 to 1.2 meq/L. Because the therapeutic effect of lithium may not appear until after 7 to 10 days of treatment, adjunctive usage of lorazepam (1 to 2 mg every 4 h) or clozepam (0.5 to 1 mg every 4 h) may be beneficial to control agitation. Antipsychotics are indicated in patients with severe agitation who respond only partially to benzodiazepines.

Valproic acid is an alternative in patients who cannot tolerate lithium or respond poorly to it. Valproic acid may be better than lithium for patients who experience rapid cycling (i.e., more than four episodes a year) or who present with a mixed or dysphoric mania. Tremor and weight gain are the most common side effects; hepatotoxicity and pancreatitis are rare toxicities. Carbamazepine and oxcarbazepine, although not formally approved by the U.S. Food and Drug Administration (FDA) for bipolar disorder, have clinical efficacy in the treatment of acute mania. Preliminary evidence also suggests that other anticonvulsant agents such as levtiracetam, zonisamide and topiramate may possess some therapeutic benefit.

The recurrent nature of bipolar mood disorder necessitates maintenance treatment. Compliance is frequently an issue and often requires enlistment and education of concerned family members. Efforts to identify and modify psychosocial factors that may trigger episodes are important, as is an emphasis on lifestyle regularity. Antidepressant medications are sometimes required for the treatment of severe breakthrough depressions, but their use should generally be avoided during maintenance treatment because of the risk of precipitating mania or accelerating the cycle frequency. Loss of efficacy over time may be observed with any of the mood-stabilizing agents. In such situations, an alternative agent or therapy is usually helpful.

There remains a need to provide an effective treatment for mania and/or for bipolar disorder. Preferably, the treatment of bipolar disorder comprises treatment of the depression and the mania. More preferably, the treatment of bipolar disorder comprises treatment of the depression, the mania and the cycling that are characteristic of the disorder.

### SUMMARY OF THE INVENTION

The present invention is directed to a method for the treatment of treatment of mania and/or bipolar disorder comprising administering to a subject in need thereof a therapeutically effective amount of a compound of formula (I)

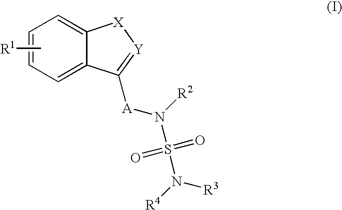

wherein

R¹ is selected from the group consisting of hydrogen, halogen, hydroxy, methoxy, trifluoromethyl, nitro and cyano;

X-Y is selected from the group consisting of —S—CH—, —S—C(CH₃)—, —O—CH—, —O—C(CH₃)—, —N(CH₃)—CH— and —CH═CH—CH—;

A is selected from the group consisting of —CH₂— and —CH(CH₃)—;

R² is selected from the group consisting of hydrogen and methyl;

R³ and R⁴ are each independently selected from the group consisting of hydrogen and C₁₋₄alkyl;

alternatively, R³ and R⁴ are taken together with the nitrogen atom to which they are bound to form a 5 to 7 membered, saturated, partially unsaturated or aromatic ring structure, optionally containing one to three additional heteroatoms independently selected from the group consisting of O, N and S;

or a pharmaceutically acceptable salt thereof.

The present invention is further directed to a method for the treatment of mania comprising co-therapy with a therapeutically effective amount of at least one antipsychotic and a compound of formula (I) as described herein. The present invention is further directed to a method for the treatment of bipolar disorder comprising co-therapy with a therapeutically effective amount of at least one antidepressant and/or at least one antipsychotic and a compound of formula (I)) as described herein. The present invention is further directed to a method for the treatment of bipolar disorder comprising co-therapy with a therapeutically effective amount of at least one mood stabilizer and a compound of formula (I) as described herein.

Exemplifying the invention is a method of treating mania comprising administering to a subject in need thereof a therapeutically effective amount of any of the compounds described above. Exemplifying the invention is a method of treating bipolar disorder comprising administering to a subject in need thereof a therapeutically effective amount of any of the compounds described above.

In an embodiment, the present invention is directed to the treatment of mania. In another embodiment, the present invention is directed to the treatment of bipolar mania. In another embodiment, the present invention is directed to the treatment of bipolar depression. In another embodiment, the present invention is directed to the treatment of bipolar disorder. In another embodiment, the present invention is directed to the treatment of the bipolar cycling. In another embodiment, the present invention is directed to the treatment of the depression and the mania associated with bipolar disorder. In yet another embodiment, the present invention is directed to the treatment of the depression, the mania and the cycling associated with bipolar disorder. In yet another embodiment, the present invention is directed to a method for treating bipolar disorder comprising stabilization of cycling. Thus, in an embodiment, the present invention is directed to a method of stabilizing bipolar cycling.

### BRIEF DESCRIPTION OF THE FIGURES

FIG. 1 illustrates the decrease in dominance of Compound #1 treated rats in the Dominant-Submissive Assay described in Example 17.

FIG. 2 illustrates historical data on the effect of water, lithium, carbamazepine and sodium valproate on dominance levels in rats tested in the Dominant-Submissive Assay described in Example 17.

FIG. 3 illustrates the effect of Compound #1 versus vehicle on amygdala kindling, as measured according the procedure described in Example 19.

FIG. 4 illustrates effect of the Compound #1 on the behavioral seizure score/development of generalized seizure in animals tested according to the procedure described in Example 19/

### DETAILED DESCRIPTION OF THE INVENTION

The present invention is directed to a method for the treatment of mania and/or bipolar disorder comprising administering to a subject in need thereof a therapeutically effective amount of a compound of formula (I)

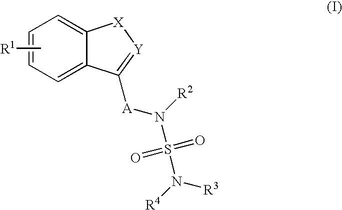

or a pharmaceutically acceptable salt thereof, wherein R¹, R², R³, R⁴, —X—Y— and A are as herein defined. More specifically, the compounds of the present invention are useful for the treatment of the mania, regardless of cause. Further, the compounds of the present invention are useful for the treatment of depression, mania and/or the cycling that are characteristic of, symptomatic of or associated with bipolar disorder.

The present invention is further directed to methods for the treatment of mania, bipolar depression, bipolar mania, bipolar cycling and/or bipolar disorder comprising administering to a subject in need thereof co-therapy with at least one antidepressant and/or at least one antipsychotic agent and/or at least one mood stabilizer and a compound of formula (I) or formula (II) as described herein.

Bipolar disorder is psychiatric disorder characterized by unpredictable swings in mood from mania (or hypomania) to depression. As used herein, the term “bipolar disorder” shall include bipolar disorder I, bipolar disorder II, cyclothymic disorder and bipolar disorder not otherwise specified. Preferably, the bipolar disorder is characterized by depressive and manic (or hypomanic) phases, wherein the phases cycle. Preferably, the bipolar disorder is bipolar disorder I or bipolar disorder II.

As used herein, the term “mania” shall include mania or a manic mood phase, regardless of underlying cause. As used herein, the term “bipolar mania” is intended to mean the mania associated with, characteristic of or symptomatic of a bipolar disorder. Thus, methods of treating bipolar mania of the present invention are directed to methods which treat the mania and/or manic phase of bipolar disorders.

As used herein, the term “bipolar depression” is intended to mean the depression associated with, characteristic of or symptomatic of a bipolar disorder. Thus, methods of treating bipolar depression of the present invention are directed to methods which treat the depression and/or depressed phase of bipolar disorders.

As used herein, unless otherwise noted the terms “cycling” or “bipolar cycling” shall refer to the alternation of mood between depressive and manic phases characteristic of bipolar disorders. Thus, the present invention includes methods for the stabilization of said cycling, including, but not limited to, decreasing the frequency of the cycling and/or decreasing the magnitude of the manic and/or depressive phases.

As used herein, the term “mood stabilizer” shall include any pharmaceutical agent which controls mood including, but not limited to, lithium, valproic acid, sodium valproate, carbamazepine, lamotrigine, topiramate, and the like. More specifically, a mood stabilizer is any pharmaceutical agent which stabilizes the patients mood may act as an antidepressant, an antimanic or both and biases the patient mood toward euthymia.

As used herein, unless otherwise noted, the term “antidepressant” shall mean any pharmaceutical agent which treats depression. Suitable examples include, but are not limited to mono-amine oxidase inhibitors such as phenelzine, tranylcypromine, moclobemide, and the like; tricyclics such as imipramine, amitriptyline, desipramine, nortriptyline, doxepin, protriptyline, trimipramine, chlomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, fluvoxamine, and the like; serotonin receptor antagonists such as nefazadone, and the like; serotonin noradrenergic reuptake inhibitors such as venlafaxine, milnacipran and the like; noradrenergic and specific serotonergic agents such as mirtazapine, and the like; noradrenaline reuptake inhibitors such as reboxetine, and the like; atypical antidepressants such as bupropion, and the like; natural products such as Kava-Kava, St. John's Wort, and the like; dietary supplements such as s-adenosylmethionine, and the like; and neuropeptides such as thyrotropin-releasing hormone and the like, and the like; compounds targeting neuropeptide receptors such as neurokinin receptor antagonists and the like; and hormones such as triiodothyronine, and the like. Preferably, the antidepressant is selected from the group consisting of fluoxetine, imipramine, bupropion, venlafaxine and sertaline.

As used herein the term “antipsychotic” is intended to includes, but are is not limited to (a) typical or traditional antipsychotics, such as phenothiazines (eg, chlorpromazine, thioridazine, fluphenazine, perphenazine, trifluoperazine, levomepromazin), thioxanthenes (eg, thiothixene, flupentixol), butyrophenones (eg, haloperidol), dibenzoxazepines (eg, loxapine), dihydroindolones (eg, molindone), substituted benzamides (eg, sulpride, amisulpride), and the like; and (b) atypical antipsychotics, such as divalproate sodium, paliperidone, clozapine, risperidone, olanzapine, quetiapine, zotepine, ziprasidone, iloperidone, perospirone, blonanserin, sertindole, ORG-5222 (Organon), and the like; and others such as sonepiprazole, aripiprazole, nemonapride, SR-31742 (Sanofi), CX-516 (Cortex), SC-111 (Scotia), NE-100 (Taisho), and the like.

More specifically, atypical antipsychotics include, but are not limited to:

2-methyl-4-(4-methyl-1-piperazinyl)-10H-thieno[2,3-b]benzodiazepine, known as olanzapine and described in U.S. Pat. No. 5,229,382 as useful for the treatment of schizophrenia, schizophreniform disorder, acute mania, mild anxiety states and psychosis; with a recommended dosage of 5-30 mg/day, preferably 5-10 mg/day (Physician's Desk Reference; Kaplan & Sadock's Comprehensive Textbook of Psychiatry, Seventh Edition, Volume II, Lippincott Williams & Wilkins: Philadelphia, 2000);

8-chloro-11-(4-methyl-1-piperazinyl)-5H-dibenzo[b,e][1,4]diazepine, known as clozapine and disclosed in U.S. Pat. No. 3,539,573, with clinical efficacy in the treatment of schizophrenia described in Hanes, et al., Psychopharmacological Bulletin, 24, 62 (1988)); with a recommended dosage of 12.5-600 mg/day, preferably 250-450 mg/day (Physician's Desk Reference; Kaplan & Sadock's Comprehensive Textbook of Psychiatry, Seventh Edition, Volume II, Lippincott Williams & Wilkins: Philadelphia, 2000);

3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one, known as risperidone and described in U.S. Pat. No. 4,804,663 as useful for the treatment of psychotic diseases; with a recommended dosage of 0.25-16 mg/day, preferably 1-16 mg/day, more preferably 2-8 mg/day (Physician's Desk Reference; Kaplan & Sadock's Comprehensive Textbook of Psychiatry, Seventh Edition, Volume II, Lippincott Williams & Wilkins: Philadelphia, 2000);

3-[2-[4-(6-fluoro-1,2-benzisoxazol-3-yl)-1-piperidinyl]ethyl]-6,7,8,9-tetrahydro-9-hydroxy-2-methyl-H-Pyrido[1,2-a]pyrimidin-4-one, known as paliperidone, also known as 9-hydroxy-risperidone, described in U.S. Pat. No. 5,5158,952, useful for the treatment of psychotic disorders, with contemplated dosages in the range of 0.01 mg/kg to about 2 mg/kg body weight per day;

1-[2-[3-[5-chloro-1-(4-fluorophenyl)-1H-indol-3-yl]-1-piperidinyl]ethyl]imidazolidin-2-one, known as sertindole and disclosed in U.S. Pat. No. 4,710,500, with U.S. Pat. No. 5,112,838 and U.S. Pat. No. 5,238,945 disclosing the use of sertindole for the treatment of schizophrenia; with a starting dose of 4 mg/day, with increases of 4 mg every other day up to 24 mg/day, with final recommended dosage range of 12 to 20 mg/day (Kaplan & Sadock's Comprehensive Textbook of Psychiatry, Seventh Edition, Volume II, Lippincott Williams & Wilkins: Philadelphia, pp. 2467-2468, 2000);

5-[2-(4-dibenzo[b,f][1,4]thiazepin-11-yl-1-piperazinyl)ethoxy]ethanol, known as quetiapine and disclosed in U.S. Pat. No. 4,879,288 for the treatment of schizophrenia; with a recommended dosage of 25-800 mg/day, preferably 150-750 mg/day (Physician's Desk Reference; Kaplan & Sadock's Comprehensive Textbook of Psychiatry, Seventh Edition, Volume II, Lippincott Williams & Wilkins: Philadelphia, 2000);

5-[2-[4-(1,2-dibenzoisothiazol-3-yl)-1-piperazinyl]ethyl]-6-chloro-1,3-dihydro-2H-indol-2-one, known as ziprasidone and disclosed in U.S. Pat. No. 4,831,031 and U.S. Pat. No. 5,312,925, with its utility in the treatment of schizophrenia disclosed in U.S. Pat. No. 4,831,031; with a recommended dosage of 40-160 mg/day, with a preferred dosage for maintenance treatment and prevention of relapse of 40 to 60 mg twice a day (Kaplan & Sadock's Comprehensive Textbook of Psychiatry, Seventh Edition, Volume II, Lippincott Williams & Wilkins: Philadelphia, pp. 2470-2471, 2000); and
sodium hydrogen bis(2-propylpentanoate), also known as divalproex sodium, described in U.S. Pat. No. 5,212,326, with a recommended dosage for the treatment of mania at an initial 750 mg/day with a maximum recommended dosage of 60 mg/kg/day (Physicians Desk Reference).

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

Wherein the present invention is directed to co-therapy or combination therapy, comprising administration of one or more compound(s) of formula (I) or formula (II) and one or more antipsychotic and/or antidepressant, “therapeutically effective amount” shall mean that amount of the combination of agents taken together so that the combined effect elicits the desired biological or medicinal response. For example, the therapeutically effective amount of co-therapy comprising administration of a compound of formula (I) or formula (II) and at least on antidepressant and/or at least one antipsychotic would be the amount of the compound of formula (I) or formula (II) and the amount of the antidepressant and/or antipsychotic that when taken together or sequentially have a combined effect that is therapeutically effective. Further, it will be recognized by one skilled in the art that in the case of co-therapy with a therapeutically effective amount, as in the example above, the amount of the compound of formula (I) or formula (II) and/or the amount of the antidepressant and/or antipsychotic individually may or may not be therapeutically effective.

As used herein, the terms “co-therapy” and “combination therapy” shall mean treatment of a subject in need thereof by administering one or more compounds of formula (I) or formula (II) in combination with one or more antidepressant(s) and/or antipsychotic(s), wherein the compound(s) of formula (I) or formula (II) and the antidepressant(s) and/or antipsychotic(s)are administered by any suitable means, simultaneously, sequentially, separately or in a single pharmaceutical formulation. Where the compound(s) of formula (I) or formula (II) and the antidepressant(s) and/or antipsychotic(s) are administered in separate dosage forms, the number of dosages administered per day for each compound may be the same or different. The compound(s) of formula (I) or formula (II) and the antidepressant(s) and/or antipsychotic(s) may be administered via the same or different routes of administration. Examples of suitable methods of administration include, but are not limited to, oral, intravenous (iv), intramuscular (im), subcutaneous (sc), transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The compound(s) of formula (I) or formula (II) and the antidepressant(s) and/or antipsychotic(s) may be administered according to simultaneous or alternating regimens, at the same or different times during the course of the therapy, concurrently in divided or single forms.

In an embodiment, the present invention is directed to a method for the treatment of depression associated with or characteristic of or symptomatic of bipolar disorder. In another embodiment, the present invention is directed to a method for the treatment of mania associated with or characteristic of or symptomatic of bipolar disorder. In yet another embodiment, the present invention is directed to a method for the treatment of cycling (between depression and mania or the depressive and manic phases) associated with or characteristic of or symptomatic of bipolar disorder.

In an embodiment of the present invention, the compound of formula (I) is selected from the group wherein

R¹ is selected from the group consisting of hydrogen, halogen, hydroxy, methoxy, trifluoromethyl, nitro and cyano;

X—Y is selected from the group consisting of —S—CH—, —S—C(CH₃)—, —O—CH—, —O—C(CH₃)—, —N(CH₃)—CH— and —CH═CH—CH—;

A is selected from the group consisting of —CH₂— and —CH(CH₃)—;

R² is selected from the group consisting of hydrogen and methyl;

R³ and R⁴ are each independently selected from the group consisting of hydrogen and methyl;

alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;

or a pharmaceutically acceptable salt thereof.

In another embodiment of the present invention, the compound of formula (I) is selected from the group wherein

R¹ is selected from the group consisting of hydrogen and halogen;

X—Y is selected from the group consisting of —S—CH—, —S—C(CH₃)—, —O—CH—, —O—C(CH₃)—, —N(CH₃)—CH— and —CH═CH—CH—;

A is selected from the group consisting of —CH₂— and —CH(CH₃)—;

R² is selected from the group consisting of hydrogen and methyl;

R³ and R⁴ are each independently selected from the group consisting of hydrogen and methyl;

and pharmaceutically acceptable salts thereof.

In another embodiment of the present invention, the compound of formula (I) is selected from the group wherein

R¹ is selected from the group consisting of hydrogen and halogen; wherein the halogen is bound at the 4-, 5- or 7-position;

X—Y is selected from the groups consisting of —O—CH—, —O—C(CH₃)—, —S—CH—, —S—C(CH₃)—, —N(CH₃)—CH— and —CH═CH—CH—;

A is selected from the group consisting of —CH₂— and —CH(CH₃)—;

R² is hydrogen;

R³ and R⁴ are each hydrogen;

and pharmaceutically acceptable salts thereof.

In another embodiment of the present invention, the compound of formula (I) is selected from the group wherein

R¹ is hydrogen;

X—Y is selected from the groups consisting of —O—CH—, —O—C(CH₃)—, —S—CH—, —S—C(CH₃)—, —N(CH₃)—CH— and —CH═CH—CH—;

A is selected from the group consisting of —CH₂— and —CH(CH₃)—;

R² is hydrogen;

R³ and R⁴ are each hydrogen;

and pharmaceutically acceptable salts thereof.

In another embodiment of the present invention, the compound of formula (I) is selected from the group wherein
R¹ is selected from the group consisting of hydrogen halogen, hydroxy, methoxy, trifluoromethyl, nitro and cyano; preferably, R¹ is selected from the group consisting of hydrogen and halogen; more preferably, R¹ is selected from the group consisting of hydrogen and halogen, wherein the halogen is bound at the 4-, 5- or 7-position;

X—Y is —S—CH—;

A is selected from the group consisting of —CH₂— and —CH(CH₃)—;

R² is selected from the group consisting of hydrogen and methyl; preferably, R² is hydrogen;

R³ and R⁴ are each independently selected from the group consisting of hydrogen and halogen; preferably, R³ and R⁴ are each hydrogen;

and pharmaceutically acceptable salts thereof.

In an embodiment of the present invention R¹ is selected from the group consisting of hydrogen, chloro, fluoro and bromo. In another embodiment of the present invention, the R¹ group is other than hydrogen and bound at the 4-, 5- or 7-position, preferably at the 5-position. In yet another embodiment of the present invention, the R¹ group is other than hydrogen and bound at the 5-, 6- or 8-position, preferably at the 6-position. In yet another embodiment of the present invention, R¹ is selected from the group consisting of hydrogen and halogen. In yet another embodiment of the present invention, R¹ is selected from the group consisting of hydroxy and methoxy. In yet another embodiment of the present invention, R¹ is selected from the group consisting of hydrogen, halogen and trifluoromethyl. In yet another embodiment of the present invention, R¹ is selected from the group consisting of hydrogen, halogen, trifluoromethyl, cyano and nitro. In yet another embodiment of the present invention, R¹ is selected from the group consisting of hydrogen, halogen, trifluoromethyl and cyano. In yet another embodiment of the present invention, R¹ is selected from the group consisting of trifluoromethyl and cyano. In yet another embodiment of the present invention, R1 is selected from the group consisting of hydrogen, 4-bromo, 5-chloro, 5-fluoro, 5-bromo, 5-trifluoromethyl-5-cyano and 7-cyano.

In an embodiment of the present invention R² is hydrogen. In another embodiment of the present invention R³ and R⁴ are each hydrogen. In yet another embodiment of the present invention R² is hydrogen, R³ is hydrogen and R⁴ is hydrogen.

In an embodiment of the present invention, R³ and R⁴ are each independently selected from the group consisting of hydrogen and C₁₋₄alkyl. In another embodiment of the present invention, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S.

In an embodiment of the present invention, R³ and R⁴ are each independently selected from the group consisting of hydrogen, methyl and ethyl. In another embodiment of the present invention, R³ and R⁴ are each independently selected from the group consisting of hydrogen and methyl. In yet another embodiment of the present invention, R³ and R⁴ are each independently selected from the group consisting of hydrogen and ethyl. In yet another embodiment of the present invention, R³ is hydrogen and R⁴ is ethyl.

In an embodiment of the present invention R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, S and N. In another embodiment of the present invention R³ and R⁴ are taken together with the nitrogen atom to which they are bound to form a 5 to 7 membered saturated ring structure, optionally containing one to two additional heteroatoms independently selected from the group consisting of O, S and N. In another embodiment of the present invention R³ and R⁴ are taken together with the nitrogen atom to which they are bound to form a 5 to 7 membered aromatic ring structure, optionally containing one to two additional heteroatoms independently selected from the group consisting of O, S and N.

Preferably, R³ and R⁴ are taken together with the nitrogen atom to which they are bound to form a 5 to 6 membered saturated, partially unsaturated or aromatic ring structure, optionally containing one to two additional heteroatoms independently selected from the group consisting of O, S and N. More preferably, R³ and R⁴ are taken together with the nitrogen atom to which they are bound to form a 6 membered saturated, partially unsaturated or aromatic ring structure, optionally containing one to two additional heteroatoms independently selected from the group consisting of O, S and N.

Preferably, R³ and R⁴ are taken together with the nitrogen atom to which they are bound to form a 5 to 7 (more preferably 5 to 6) membered saturated or aromatic ring structure, optionally containing one to two (preferably one) additional heteroatoms independently selected from the group consisting of O, S and N (preferably O or N, more preferably N).

In another embodiment of the present invention, R³ and R⁴ are taken together with the nitrogen atom to which they are bound to form a 5 to 6 membered saturated or aromatic ring structure, optionally containing one to two (preferably one) additional heteroatoms independently selected from the group consisting of O, S and N (preferably O or N, more preferably, N).

Preferably, the 5 to 7 membered saturated, partially unsaturated or aromatic ring structure contains 0 to 1 additional heteroatoms independently selected from the group consisting of O, S and N. Preferably, the heteroatom is independently selected from the group consisting of O and N, more preferably, the heteroatom is N.

Suitable examples of the 5 to 7 membered, saturated, partially unsaturated or aromatic ring structures which optionally contain one to two additional heteroatoms independently selected from the group consisting of O, S and N include, but are not limited to pyrrolyl, pyrrolidinyl, pyrrolinyl, morpholinyl, piperidinyl, piperazinyl, imidazolyl, pyrazolyl, pyridyl, imidazolyl, thiomorpholinyl, pyrazinyl, triazinyl, azepinyl, and the like. Preferred 5 to 7 membered, saturated, partially unsaturated or aromatic ring structures which optional containing one to two additional heteroatoms independently selected from the group consisting of O, S and N include, but are not limited, to imidazolyl, pyrrolidinyl, piperidinyl and morpholinyl.

In an embodiment of the present invention A is —CH₂—.

In an embodiment of the present invention X—Y is selected from the group consisting of —S—CH—, —O—CH—, —O—C(CH₃)—, —N(CH₃)—CH— and —CH═CH—CH—. In another embodiment of the present invention X—Y is selected from the group consisting of —S—CH—, —O—CH—, —O—C(CH₃)— and —CH═CH—CH—. In yet another embodiment of the present invention X—Y is selected form the group consisting of —S—CH—, —O—CH—, —O—C(CH₃)— and —N(CH₃)—CH—. In yet another embodiment of the present invention X—Y is selected from the group consisting of —S—CH—, —O—CH—, —N(CH₃)—CH— and —CH═CH—CH—. In yet another embodiment of the present invention X—Y is selected from the group consisting of —S—CH—, —O—CH— and —CH═CH—C—. In yet another embodiment of the present invention, X—Y is selected from the group consisting of —S—CH— and —O—CH—. In yet another embodiment of the present invention, X—Y is selected from the group consisting of S—CH—, —S—C(CH₃)—, —O—CH—, —O—C(CH₃)— and —N(CH₃)—CH—.

In an embodiment of the present invention, X— is —S—CH—. In another embodiment of the present invention X—Y is —CH═CH═CH—. In yet another embodiment of the present invention X—Y is —N(CH₃)—CH—. In yet another embodiment of the present invention X—Y is selected from the group consisting of —O—CH— and —O—C(CH₃)—.

In an embodiment, the present invention is directed to a compounds selected from the group consisting of N-(benzo[b]thien-3-ylmethyl)-sulfamide; N-[(5-chlorobenzo[b]thien-3-yl)methyl]-sulfamide; N-(3-benzofuranyl methyl)-sulfamide; N-[(5-fluorobenzo[b]thien-3-yl)methyl]-sulfamide; N-(1-benzo[b]thien-3-ylethyl)-sulfamide; N-(1-naphthalenylmethyl)-sulfamide; N-[(2-methyl-3-benzofuranyl)methyl]-sulfamide; N-[(5-bromobenzo[b]thien-3-yl )methyl]-sulfamide; N-[(4-bromobenzo[b]thien-3-yl)methyl]-sulfamide; N-[(7-fluorobenzo[b]thien-3-yl)methyl]-sulfamide; N-[(1-methyl-1H-indol-3-yl)methyl]-sulfamide; N-[(4-trifluoromethylbenzo[b]thien-3-yl )methyl]-sulfamide; N-[(4-cyanobenzo[b]thien-3-yl )methyl]-sulfamide; N-[(benzo[b]thien-3-yl)methyl]-sulfamoylpyrrolidine; N-[(benzo[b]thien-3-yl)methyl]-N′-ethylsulfamide; Imidazole-1-sulfonic acid [(benzo[b]thien-3-yl)methyl]-amide; and pharmaceutically acceptable salts thereof.

Additional embodiments of the present invention, include those wherein the substituents selected for one or more of the variables defined herein (i.e. R¹, R², R³, R⁴, X—Y and A) are independently selected to be any individual substituent or any subset of substituents selected from the complete list as defined herein.

Representative compounds useful in the treatment of depression are as listed in Table 1 and 2, below.

TABLE 1
Representative Compounds of Formula (I)

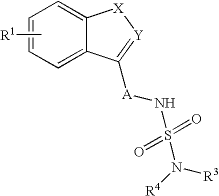

| ID No. | R¹ | —X—Y— | A | R³ | R⁴ |
|---|---|---|---|---|---|
| 1 | H | —S—CH— | —CH₂— | H | H |
| 3 | 5-Cl | —S—CH— | —CH₂— | H | H |
| 6 | H | —O—CH— | —CH₂— | H | H |
| 7 | H | —N(CH₃)—CH— | —CH₂— | H | H |
| 8 | 5-F | —S—CH— | —CH₂— | H | H |
| 9 | H | —S—CH— | —CH(CH₃)— | H | H |
| 10 | H | —CH═CH—CH— | —CH₂— | H | H |
| 13 | H | —O—C(CH₃) | —CH₂— | H | H |
| 15 | 5-Br | —S—CH— | —CH₂— | H | H |
| 17 | 4-Br | —S—CH— | —CH₂— | H | H |
| 18 | 7-F | —S—CH— | —CH₂— | H | H |
| 19 | 5-CF₃ | —S—CH— | —CH₂— | H | H |
| 20 | 5-CN | —S—CH— | —CH₂— | H | H |
| 21 | H | —S—CH— | —CH₂— | H | ethyl |

TABLE 2

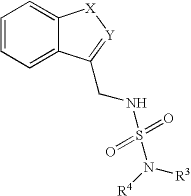

| ID No. | —X—Y— | R3 + R4 together with the N atom |
|---|---|---|
| 101 | —S—CH— | N-pyrrolidinyl |
| 102 | —S—CH— | N-imidazolyl |

As used herein, “halogen” shall mean chlorine, bromine, fluorine and iodine.

As used herein, the term “alkyl” whether used alone or as part of a substituent group, include straight and branched chains. For example, alkyl radicals include methyl, ethyl, propyl, isopropyl, butyl, isobutyl, sec-butyl, t-butyl, pentyl and the like. Unless otherwise noted, “C₁₋₄alkyl” means a carbon chain composition of 1-4 carbon atoms.

When a particular group is “substituted” (e.g., alkyl, phenyl, aryl, heteroalkyl, heteroaryl), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

Unless otherwise noted, the position at which the R¹ substituent is bound will be determined by counting around the core structure in a clockwise manner beginning at the X—Y positions as 1,2 and continuing from thereon as follows:

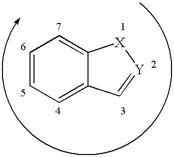

Should the X—Y substituent be —CH═CH—CH—, then the X—Y group will be counted as 1, 2, 3 and counting then continued clockwise around the core structure as previously noted.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

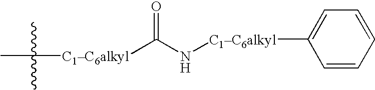

Abbreviations used in the specification, particularly the Schemes and Examples, are as follows:

DCE=Dichloroethane

DCM=Dichloromethane

DMF=N,N-Dimethylformamide

DMSO=Dimethylsulfoxide

LAH=Lithium Aluminum Hydride

MTBE=Methyl-tert-butyl ether

THF=Tetrahydrofuran

TLC=Thin Layer Chromatography

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following:

acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following:

acids including acetic acid, 2,2-dichlorolactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitric acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and

bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Compounds of formula (I) wherein A is —CH₂— may be prepared according to the process outlined in Scheme 1.

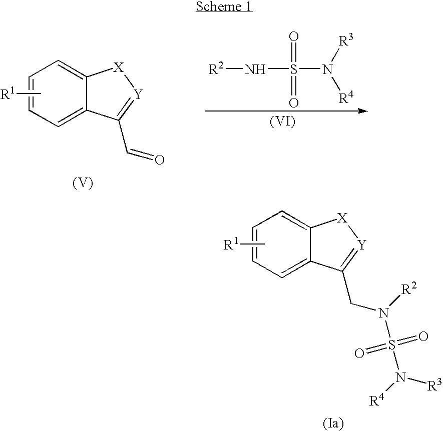

Accordingly, a suitably substituted compound of formula (V), a known compound or compound prepared by known methods, is reacted with a suitably substituted compound of formula (VI), a known compound or compound prepared by known methods, wherein the compound of formula (VI) is present in an amount in the range of about 2 to about 5 equivalents, in an organic solvent such as ethanol, methanol, dioxane, and the like, preferably, in an anhydrous organic solvent, preferably, at an elevated temperature in the range of about 50° C. to about 100° C, more preferably at about reflux temperature, to yield the corresponding compound of formula (Ia).

Compounds of formula (I) may alternatively be prepared according to the process outlined in Scheme 2.

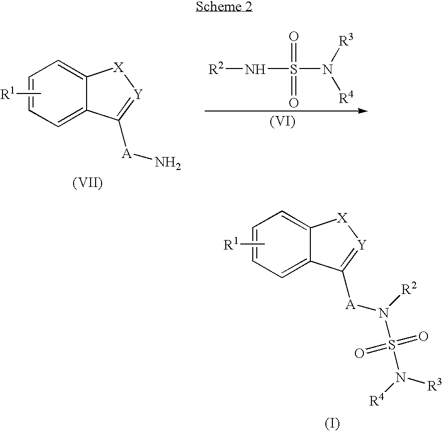

Accordingly, a suitably substituted compound of formula (VII), a known compound or compound prepared by known methods, is reacted with a suitably substituted compound of formula (VI), a known compound or compound prepared by known methods, wherein the compound of formula (VI) is present in an amount in the range of about 2 to about 5 equivalents, in an organic solvent such as THF, dioxane, and the like, preferably, in an anhydrous organic solvent, preferably, at an elevated temperature in the range of about 50° C. to about 100° C., more preferably at about reflux temperature, to yield the corresponding compound of formula (I).

Compounds of formula (VII) wherein A is —CH₂— may, for example, be prepared by according to the process outlined in Scheme 3.

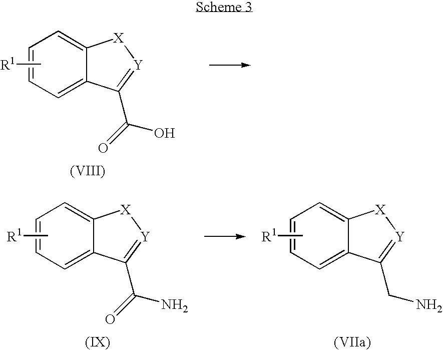

Accordingly, a suitably substituted a compound of formula (VIII), a known compound or compound prepared by known methods is reacted with an activating agent such as oxalyl chloride, sulfonyl chloride, and the like, and then reacted with an amine source such as ammonia, ammonium hydroxide, and the like, in an organic solvent such as THF, diethyl ether, DCM, DCE, and the like, to yield the corresponding compound of formula (IX).

The compound of formula (IX) is reacted with a suitably selected reducing agent such as LAH, borane, and the like, in an organic solvent such as THF, diethyl ether, and the like, to yield the corresponding compound of formula (VIIa).

Compounds of formula (VII) wherein A is —CH(CH₃)— may, for example, be prepared according to the process outlined in Scheme 4.

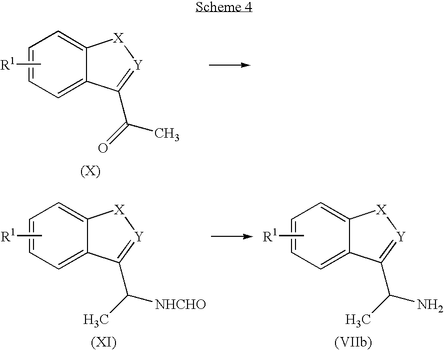

Accordingly, a suitably substituted compounds of formula (X), a known compound or compound prepared by known methods, is reacted with a mixture of formamide and formic acid, wherein the mixture of formamide and formic acid is present in an amount greater than about 1 equivalent, preferably, in an excess amount of greater than about 5 equivalent, at an elevated temperature of about 150° C., to yield the corresponding compound of formula (XI).

The compound of formula (XI) is hydrolyzed by reacting with concentrated HCl, concentrated H₂SO₄, and the like, at an elevated temperature, preferably at reflux temperature, to yield the corresponding compound of formula (VIIb).

Compounds of formula (VII) may alternatively, be prepared according to the process outlined in Scheme 5.

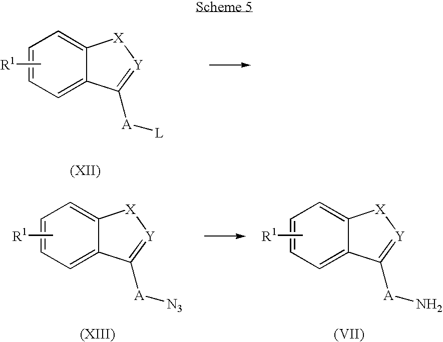

Accordingly, a suitably substituted compound of formula (XII), wherein L is a leaving group such as Br, Cl, I, tosylate, mesylate, and the like, a known compound or compound prepared by known methods, is reacted with sodium azide, in an organic solvent such a DMF, DMSO, methanol, ethanol, and the like, to yield the corresponding compound of formula (XIII).

The compound of formula (XIII) is reacted with a suitably selected reducing agent such as LAH, triphenylphosphine, H_{2(g)}, and the like, according to known methods, to yield the corresponding compound of formula (VII).

Compounds of formula (VII) wherein A is CH₂ and X—Y is —O—CH₂— may, for example, be prepared according to the process outlined in Scheme 6.

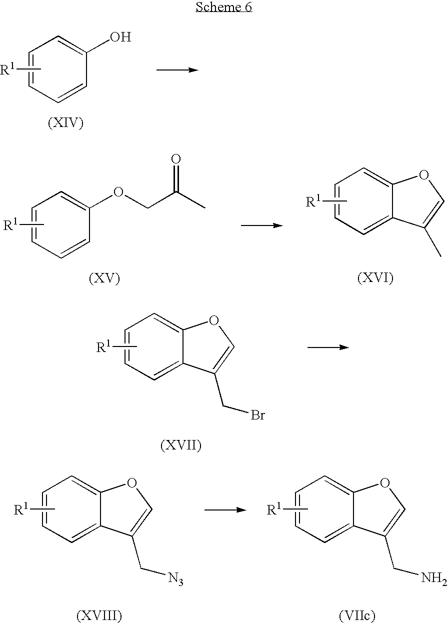

Accordingly, a suitably substituted phenol, a compound of formula (XIV), a known compound or compound prepared by known methods is reacted with bromoacetone, a known compound, in the presence of a base such as K₂CO₃, Na₂CO₃, NaH, triethylamine, pyridine, and the like, in an organic solvent such as acetonitrile, DMF, THF, and the like, optionally at an elevated temperature, to yield the corresponding compound of formula (XV).

The compound of formula (XV) is reacted with an acid such as polyphosphoric acid, sulfuric acid, hydrochloric acid, and the like, preferably with polyphosphoric acid, preferably in the absence of a solvent (one skilled in the art will recognize that the polyphosphoric acid acts as the solvent), to yield the corresponding compound of formula (XVI).

The compound of formula (XVI) is reacted with a source of bromine such as N-bromosuccinimide in the presence of benzoylperoixde, Br₂, and the like, in an organic solvent such as carbon tetrachloride, chloroform, DCM, and the like, preferably in a halogenated organic solvent, to yield the corresponding compound of formula (XVII).

The compound of formula (XVII) is reacted with sodium azide, in an organic solvent such a DMF, DMSO, methanol, ethanol, and the like, to yield the corresponding compound of formula (XVIII).

The compound of formula (XVIII) is reacted with a suitably selected reducing agent such as LAH, triphenylphosphine, H_{2(g)}, and the like, according to known methods, to yield the corresponding compound of formula (VIIc).

Compounds of formula (V) wherein X—Y is —S—CH— may, for example, be prepared according to the process outlined in Scheme 7.

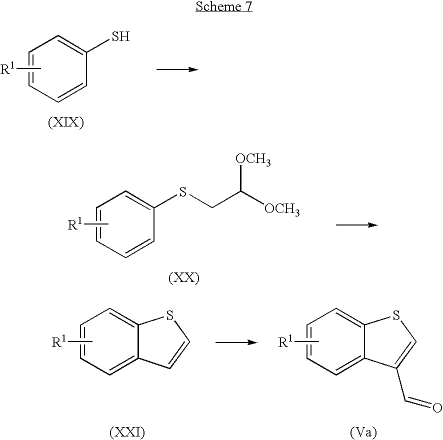

Accordingly, a suitably substituted compound of formula (XIX), a known compound or compound prepared by known methods is reacted with choroacetaldehyde dimethyl acetal or bromoacetaldehyde dimethyl acetal, a known compound, in the presence of a base such as potassium-tert-butoxide, sodium-tert-butxide, potassium carbonate, potassium hydroxide, and the like, in an organic solvent such as THF, DMF, acetonitrile, and the like, to yield the corresponding compound of formula (XX).

The compound of formula (XX) is reacted with reacted with an acid such as polyphosphoric acid, sulfuric acid, hydrochloric acid, and the like, preferably with polyphosphoric acid in the presence of chlorobenzene, preferably in the absence of a solvent (one skilled in the art will recognize that the polyphosphoric acid and/or the chlorobenzene may act as the solvent), at an elevated temperature in the range of from about 100 to 200° C., preferably at an elevated temperature of about reflux temperature, to yield the corresponding compound of formula (XXI).

The compound of formula (XXI) is reacted with a formylating reagent such as dichloromethyl methyl ether, and the like, in the presence of Lewis acid catalyst such as titanium tetrachloride, aluminum trichloride, tin tetrachloride, and the like, in an organic solvent such as DCM, chloroform, and the like, at a temperature in the range of from about 0° C. to about room temperature, to yield the corresponding compound of formula (Va).

Compounds of formula (I) wherein R³ and/or R⁴ are other than hydrogen or R³ and R⁴ are taken together with the nitrogen to which they are bound to form a ring structure, may alternatively be prepared according to the process outlined in Scheme 8.

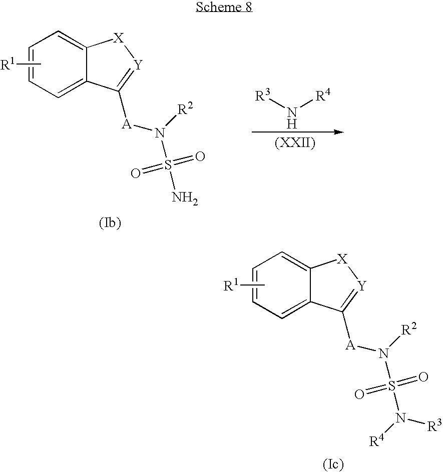

Accordingly, a suitably substituted compound of formula (Ib), is reacted with a suitably substituted amine, a compound of formula (XXII), a known compound or compound prepared by known methods, in water or an organic solvent such as dioxane, ethanol, THF, isopropanol, and the like, provide that the compound of formula (Ib) and the compound of formula (XXII) are at least partially soluble in the water or organic solvent, at a temperature in the range of from about room temperature to about reflux, preferably at about reflux temperature, to yield the corresponding compound of formula (Ic).

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.1-1000 mg and may be given at a dosage of from about 0.01-200.0 mg/kg/day, preferably from about 0.1 to 100 mg/kg/day, more preferably from about 0.5-50 mg/kg/day, more preferably from about 1.0-25.0 mg/kg/day or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 1000 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating mania and/or bipolar disorder described in the present invention may also be carried out using a pharmaceutical composition comprising any of the compounds as defined herein and a pharmaceutically acceptable carrier. The pharmaceutical composition may contain between about 0.1 mg and 1000 mg, preferably about 50 to 500 mg, of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methylcellulose and the like. For parenteral administration, sterile suspensions and solutions are desired. Isotonic preparations which generally contain suitable preservatives are employed when intravenous administration is desired.

Compounds of this invention may be administered in any of the foregoing compositions and according to dosage regimens established in the art whenever treatment of depression is required.

The daily dosage of the products may be varied over a wide range from 0.01 to 200 mg/kg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 200 mg/kg of body weight per day. Preferably, the range is from about 0.1 to about 100.0 mg/kg of body weight per day, more preferably, from about 0.5 mg/kg to about 50 mg/kg, more preferably, from about 1.0 to about 25.0 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

### EXAMPLE 1

### N-(benzo[b]thien-3-ylmethyl)-sulfamide (Compound #1)

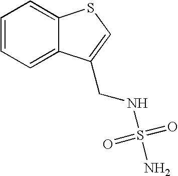

Thianaphthene-3-carboxaldehyde (1.62 g, 10.0 mmol) was dissolved in anhydrous ethanol (50 mL). Sulfamide (4.0 g, 42 mmol) was added and the mixture was heated to reflux for 16 hours. The mixture was cooled to room temperature. Sodium borohydride (0.416 g, 11.0 mmol) was added and the mixture was stirred at room temperature for three hours. The reaction was diluted with water (50 mL) and extracted with chloroform (3×75 mL). The extracts were concentrated and chromatographed (5% methanol in DCM) to yield the title compound as a white solid.

¹H NMR (DMSO-d₆): δ 7.98 (1H, dd, J=6.5, 2.3 Hz), 7.92 (1H, dd, J =6.6, 2.4 Hz), 7.62 (1H, s), 7.36-7.45 (2H, m), 7.08 (1H, t, J=6.3 Hz), 6.72 (2H, s), 4.31 (2H, d, J=6.3 Hz).

### EXAMPLE 2

### N-[(5-chlorobenzo[b]thien-3-yl)methyl]-sulfamide (Compound #3)

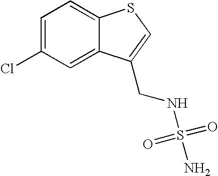

(5-Chloro-1-benzothiophene-3-yl)methylamine (0.820 g, 4.15 mmol) and sulfamide (2.5 g, 26 mmol) were combined in anhydrous dioxane (50 mL) and the mixture was heated to reflux for four hours. The reaction was cooled and diluted with water (50 mL). The solution was extracted with chloroform (3×75 mL). The extracts were concentrated and chromatographed (5% methanol in DCM) to yield the title compound as a white solid.

¹H NMR (DMSO-d₆): δ 8.05 (2H, m), 7.74 (1H, s), 7.40 (1H, d, J=6.5 Hz), 7.07 (1H, t, J=6.3 Hz), 6.72 (2H, s), 4.26 (2H, d, J=6.4 Hz).

### EXAMPLE 3

### N-[(1-methyl-1H-indol-3-yl)methyl]-sulfamide (Compound #7)

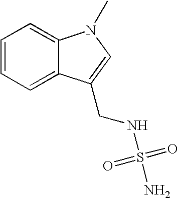

N-Methylindole-3-carboxaldehyde (1.66 g, 10.4 mmol) was dissolved in anhydrous ethanol (50 mL). Sulfamide (4.5 g, 47 mmol) was added and the mixture was heated to reflux for 16 hours. Additional sulfamide (1.0 g, 10.4 mmol) was added and the mixture was heated to reflux for 24 hours. The mixture was cooled to room temperature. Sodium borohydride (0.722 g, 12.5 mmol) was added and the mixture was stirred at room temperature for one hour. The reaction was diluted with water (50 mL) and extracted with DCM (3×75 mL). The extracts were concentrated and about 1 mL of methanol was added to create a slurry which was filtered to yield the title compound as a white powder.

¹H NMR (CD₃OD): δ 7.67 (1H, d, J=5.9 Hz), 7.32 (1H, d, J=6.2 Hz), 7.14-7.19 (2H, m), 7.06 (1H, dt, J=7.7, 0.7 Hz), 4.36 (2H, s), 3.75 (3H, s)

MS (M-H)⁻ 237.6.

### EXAMPLE 4

### N-(3-benzofuranylmethyl)-sulfamide (Compound #6)

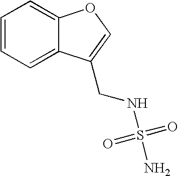

Benzofuran-3-carboxylic acid (1.91 g, 11.8 mmol) was suspended in anhydrous DCM (75 mL). Oxalyl chloride (2.0 M in DCM, 6.48 mL) and then one drop of dimethylformamide were added. The solution was stirred at room temperature for two hours, then ammonium hydroxide (concentrated, 10 mL) was added. The resulting mixture was diluted with water (100 mL) and extracted with DCM (3×100 mL). The extracts were concentrated to a gray solid and dissolved in anhydrous THF (100 mL). Lithium aluminum hydride (1.0 M in THF, 11.8 mL) was added. The mixture was stirred at room temperature for 16 hours. A minimal amount of saturated aqueous NaHCO₃ and then MgSO₄ were added. The mixture was filtered and then extracted with 1 N HCl. The aqueous extracts were adjusted to pH 14 with 3N NaOH and extracted with DCM. The organic extracts were dried with magnesium sulfate and concentrated to a colorless oil. The oil was dissolved in dioxane (50 mL) and sulfamide (3.7 g, 38 mmol) was added. The mixture was heated to reflux for 4 hours, cooled to room temperature, and concentrated. The resulting solid was chromatographed (5% methanol in DCM) to yield the title compound as a slightly yellow solid.

¹H NMR (CD₃OD): δ 7.53 (1H, d, J=5.7 Hz), 7.44 (1H, d, J=6.0 Hz), 7.16-7.26 (2H, m), 6.73 (1H, s), 4.35 (2H, s).

### EXAMPLE 5

### N-[(5-fluorobenzo[b]thien-3-yl)methyl]-sulfamide (Compound #8)

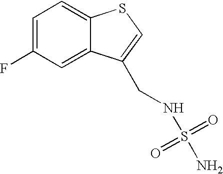

5-Fluoro-3-methylbenzothiophene (1.14 g, 6.83 mmol), benzoyl peroxide (0.165 g, 0.68 mmol) and N-bromosuccinimide (1.70 g, 7.52 mmol) were combined in carbon tetrachloride (25 mL) and the mixture was heated to reflux for 3 hours. The yellow solution was cooled, diluted with water, and extracted with DCM (2×50 mL). The extracts were washed with brine (100 mL), dried with magnesium sulfate, and concentrated to an orange solid. The solid was dissolved in anhydrous DMF. Sodium azide (4.0 g, 61 mmol) was added and the mixture was stirred for 16 hours at room temperature. The reaction was diluted with water (100 mL) and extracted with diethyl ether (2×75 mL). The extracts were washed with brine (100 mL), dried with magnesium sulfate, and concentrated to a yellow oil. The oil was dissolved in a mixture of THF (50 mL) and water (5 mL). Triphenylphosphine (3.60 g, 13.7 mmol) was added. The mixture was stirred at room temperature for 16 hours. The reaction was concentrated and chromatographed (2 to 5% methanol in DCM). The resulting C-(5-fluoro-benzo[b]thien-3-yl)-methylamine (1.04 g, 5.73 mmol) was dissolved in anhydrous dioxane (50 mL) and sulfamide (2.75 g, 28.7 mmol) was added. The reaction was heated to reflux for 4 hours, cooled to room temperature, and concentrated to a solid which was chromatographed (5% methanol in DCM) to yield the title compound as a white solid.

¹H NMR (CD₃OD): δ7.85 (1H, dd, J=6.6, 3.6 Hz), 7.66 (1H, dd, J=7.4, 1.8 Hz), 7.62 (1H, s), 7.13-7.18 (1H, m), 4.40 (2H, s).

### EXAMPLE 6

### N-(1-benzo[b]thien-3-ylethyl)-sulfamide (Compound #9)

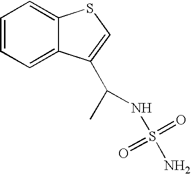

3-Acetylthianaphthene (3.00 g, 17.0 mmol) was added to a mixture of formic acid (10 mL) and formamide (10 mL). The solution was heated to 150° C. for 8 hours. The reaction was cooled to room temperature, diluted with water (50 mL), and extracted with diethyl ether (3×50 mL). The ether extracts were washed with saturated aqueous NaHCO₃ and brine. The solution was concentrated and chromatographed (5% methanol in DCM) to yield N-(1-benzo[b]thiophen-3-yl-ethyl)-formamide (1.76 g) as a white solid which was suspended in concentrated HCl (30 mL). The mixture was heated to reflux for 1.5 hours then diluted with water (100 mL). 3N NaOH was added until the pH was 14. The mixture was extracted with diethyl ether (3×100 mL) then dried with magnesium sulfate and concentrated to an orange oil. The oil was dissolved in anhydrous dioxane (75 mL) and sulfamide was added. The mixture was heated to reflux for 2 hours then diluted with water (50 ml). The solution was extracted with ethyl acetate (2×50 mL), dried with magnesium sulfate, concentrated, and chromatographed (2.5% to 5% methanol in DCM) to yield the title compound as a white solid.

¹H NMR (CD₃OD): δ 8.01 (1H, dd, J=5.5, 0.7 Hz), 7.85 (1H, dt, J=6.0, 0.6 Hz), 7.49 (1H, s), 7.31-7.40 (2H, m), 4.95 (1H, q, J=5.1 Hz), 1.67 (3H, d, J=5.1 Hz).

### EXAMPLE 7

### N-(1-naphthalenylmethyl)-sulfamide (Compound #10)

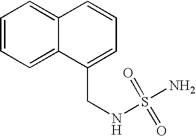

1-Naphthanlenemethylamine (2.00 g, 12.7 mmol) and sulfamide (5.0 g, 52 mmol) were combined in anhydrous dioxane (100 mL) and the mixture was heated to reflux for 6 hours. The reaction was cooled to room temperature and was filtered. The filtrate was concentrated to a solid and washed with water until TLC indicated no remaining trace of sulfamide in the solid. The collected solid was dried under vacuum to yield the title compound as a white solid.

¹H NMR (CDCl₃): δ 8.09 (1H, d, J=6.3 Hz), 7.86 (1H, dd, J=12.9, 6.2 Hz), 7.42-7.61 (4H, m), 4.75 (2H, d, J=4.4 Hz), 4.58 (1H, br s), 4.51 (2H, br s).

### EXAMPLE 8

### N-[(2-methyl-3-benzofuranyl)methyl]-sulfamide (Compound #13)

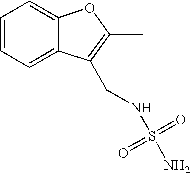

2-Methylbenzofuran-3-carbaldehyde (0.51 g, 3.18 mmol) was dissolved in anhydrous ethanol (25 mL). Sulfamide (1.5 g, 16 mmol) was added and the mixture was heated to reflux for 4 days. The mixture was cooled to room temperature. Sodium borohydride (0.132 g, 3.50 mmol) was added and the mixture was stirred at room temperature for 24 hours. The reaction was diluted with water (100 mL) and extracted with DCM (3×75 mL). The extracts were concentrated and suspended in a minimal amount of DCM and filtered to yield the title compound as a white solid.

¹H NMR (DMSO-d₆): δ 7.65 (1H, dd, J=6.4, 2.6 Hz), 7.43-7.47 (1H, m), 7.19-7.23 (2H, m), 6.87 (1H, t, J=6.2 Hz), 6.68 (2H, s), 4.11 (2H, d, J=6.2 Hz), 2.42 (3H, s).

### EXAMPLE 9

### N-[(5-bromobenzo[b]thien-3-yl)methyl]-sulfamide (Compound #15)

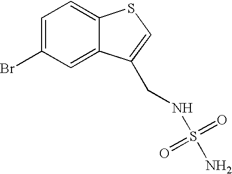

5-Bromobenzothiophene (1.60 g, 7.51 mmol) and dichloromethyl methyl ether (1.29 g, 11.3 mmol) were dissolved in anhydrous 1,2-dichloroethane (75 mL). Titanium tetrachloride (2.14 g, 11.3 mmol) was added, turning the solution dark. After one hour at room temperature, the reaction was poured into a mixture of saturated aqueous NaHCO₃ and ice. The mixture was stirred for about 30 minutes and then was extracted with DCM (2×100 mL). The extracts were concentrated and chromatographed (0 to 5% ethyl acetate in hexane) to yield 5-bromo-benzo[b]thiophene-3-carbaldehyde (1.32 g). The 5-bromobenzothiophene-3-carboxaldehyde (1.20 g, 4.98 mmol) and sulfamide (4.0 g, 42 mmol) were combined in anhydrous ethanol (25 mL) and heated to reflux for three days. The reaction was cooled to room temperature and sodium borohydride (0.207 g, 5.47 mmol) was added. After five hours, water (50 ml) was added and the solution was extracted with chloroform (3×50 mL). The extracts were concentrated, suspended in a minimal amount of DCM, and filtered to provide the title compound as a yellow solid.

¹H NMR (DMSO-d₆): δ 8.12 (1H, d, J=1.8 Hz), 7.97 (1H, d, J=8.6), 7.71 (1H, s), 7.52 (1H, dd, J=8.6, 1.9 Hz), 7.12 (1H, t, J=6.3 Hz), 6.72 (2H, s), 4.28 (2H, d, J=6.2 Hz).

### EXAMPLE 10

### N-[(4-bromobenzo[b]thien-3-yl)methyl]-sulfamide (Compound #17)

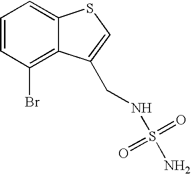

4-Bromobenzothiophene (1.80 g, 8.45 mmol) and dichloromethyl methyl ether (1.46 g, 12.7 mmol) were dissolved in anhydrous DCM (100 mL). Titanium tetrachloride (2.40 g, 12.7 mmol) was added, turning the solution dark. After 30 minutes at room temperature, the reaction was poured into a mixture of saturated aqueous NaHCO₃ and ice. The mixture was stirred for about 30 minutes and then was extracted with DCM (2×150 mL). The extracts were concentrated and chromatographed (0 to 15% ethyl acetate in hexane) to yield 4-bromobenzothiophene-3-carboxaldehyde (0.910 g). The 4-bromobenzothiophene-3-carboxaldehyde (0.910 g, 3.77 mmol) and sulfamide (3.0 g, 31 mmol) were combined in anhydrous ethanol (25 mL) and heated to reflux for three days. The reaction was cooled to room temperature and sodium borohydride (0.157 g, 4.15 mmol) was added. After five hours, water (50 ml) was added and the solution was extracted with chloroform (3×50 mL). The extracts were concentrated, suspended in a minimal amount of DCM, and filtered to yield the title compound as a yellow solid.

¹H NMR (DMSO-d₆): δ 8.05 (1H, dd, J=8.1, 0.8 Hz), 7.78 (1H, s), 7.64 (1H, dd, J=7.6, 0.8 Hz), 7.27 (1H, t, J=7.9 Hz), 7.13 (1H, t, J=6.3 Hz), 6.72 (2H, br s), 4.65 (2H, d, J=5.3 Hz).

### EXAMPLE 11

### N-[(7-fluorobenzo[b]thien-3-yl)methyl]-sulfamide (Compound #18)

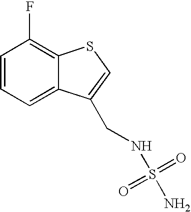

2-Fluorothiophenol (4.14 g, 32.6 mmol) was dissolved in anhydrous THF (100 mL). Potassium tert-butoxide (1.0 M in THF, 35.8 mL) was added and the suspension was stirred at room temperature for 15 minutes. 2-Chloroacetaldehyde dimethyl acetal was added and the mixture was stirred for 3 days. Water (100 mL) was added and the solution was extracted with diethyl ether (3×100 mL). The extracts were concentrated to a yellow oil and chromatographed (5 to 20% ethyl acetate in hexane) to yield 1-(2,2-dimethoxy-ethylsulfanyl)-2-fluoro-benzene (6.42 g) as a colorless oil. Chlorobenzene (25 mL) was heated to reflux and polyphosphoric acid (1 mL) was added. The 1-(2,2-dimethoxy-ethylsulfanyl)-2-fluoro-benzene was then added slowly turning the solution dark. After 3 hours of heating, the reaction was cooled to room temperature and diluted with water (50 mL). The solution was extracted with benzene (2×50 mL). The extracts were concentrated and chromatographed (0 to 15% ethyl acetate in hexane) to yield 7-fluorobenzothiophene (0.77 g). The 7-fluorobenzothiophene (0.77 g, 5.1 mmol) and dichloromethyl methyl ether (0.872 g, 7.6 mmol) were dissolved in anhydrous DCM (25 mL). Titanium tetrachloride (1.0 M in DCM, 7.6 mL, 7.6 mmol) was added, turning the solution dark. After 30 minutes at room temperature, the reaction was poured into a mixture of saturated aqueous NaHCO₃ and ice. The mixture was stirred for about 30 minutes and then was extracted with DCM (2×50 mL). The extracts were concentrated and chromatographed (0 to 15% ethyl acetate in hexane) to yield 7-fluorobenzothiophene-3-carboxaldehyde (0.642 g). The 7-fluorobenzothiophene-3-carboxaldehyde (0.642 g, 3.77 mmol) and sulfamide (1.7 g, 18 mmol) were combined in anhydrous ethanol (20 mL) and heated to reflux for three days. The reaction was cooled to room temperature and sodium borohydride (0.148 g, 3.92 mmol) was added. After two hours, water (25 ml) was added and the solution was extracted with chloroform (3×25 mL). The extracts were concentrated, suspended in a minimal amount of DCM, and filtered to yield the title compound as a yellow solid.

¹H NMR (DMSO-d₆): δ 7.78 (1H, d, J=8.0 Hz), 7.43-7.50 (1H, m), 7.27 (1H, dd, J=10.3, 7.9 Hz), 7.14 (1H, t, J=6.4 Hz), 6.74 (2H, br s), 4.31 (2H, d, J=6.4 Hz).

### EXAMPLE 12

### N-[(4-trifluoromethylbenzo[b]thien-3-yl)methyl]-sulfamide (Compound #19)

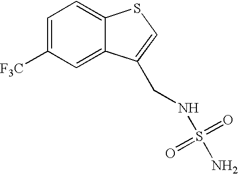

4-Trifluoromethylbenzothiophene (0.276 g, 1.37 mmol) and dichloromethyl methyl ether (0.236 g, 2.06 mmol) were dissolved in anhydrous DCM (10 mL). Titanium tetrachloride (1.0M in DCM, 2.1 mL, 2.1 mmol) was added, turning the solution dark. After 30 minutes at room temperature, the reaction was poured into a mixture of saturated aqueous NaHCO₃ and ice. The mixture was stirred for about 30 minutes and then extracted with DCM (2×25 mL). The extracts were concentrated and chromatographed (0 to 15% ethyl acetate in hexane) to yield 4-trifluoromethylbenzothiophene-3-carboxaldehyde.

The 4-trifluoromethylbenzothiophene-3-carboxaldehyde (0.226 g, 0.982 mmol) and sulfamide (0.471 g, 4.91 mmol) were combined in anhydrous ethanol (5 mL) and heated to reflux for 24 hours. The reaction was cooled to room temperature and sodium borohydride (0.056 g, 1.47 mmol) was added. After five hours, water (10 ml) was added and the solution was extracted with chloroform (3×10 mL). The extracts were concentrated, and chromatographed (5% methanol in DCM) to yield the title compound as a white solid.

¹H NMR (DMSO-d₆): δ 8.30 (1H, s), 8.25 (1H, d, J=8.4 Hz), 7.84 (1H, s), 7.68 (1H, dd, J=8.5, 1.4 Hz), 6.7-6.9 (2H, br s), 4.4-4.5 (1H, br s), 4.37 (2H, s).

### EXAMPLE 13

### N-[(4-cyanobenzo[b]thien-3-yl)methyl]-sulfamide (Compound #20)

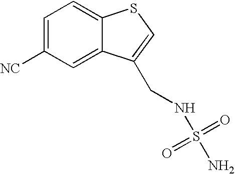

4-Cyanobenzothiophene (1.15 g, 7.22 mmol) and dichloromethyl methyl ether (1.25 g, 10.8 mmol) were dissolved in anhydrous DCM (100 mL). Titanium tetrachloride (1.0M in DCM, 10.8 mL, 10.8 mmol) was added, turning the solution dark. After 30 minutes at room temperature, the reaction was poured into a mixture of saturated aqueous NaHCO₃ and ice. The mixture was stirred for about 30 minutes and then was extracted with DCM (2×50 mL). The extracts were concentrated and chromatographed (0 to 15% ethyl acetate in hexane) to yield 4-cyanobenzothiophene-3-carboxaldehyde.

The 4-cyanobenzothiophene-3-carboxaldehyde (0.298 g, 1.59 mmol) and sulfamide (0.766 g, 7.97 mmol) were combined in anhydrous ethanol (20 mL) and heated to reflux for 24 hours. The reaction was cooled to room temperature and sodium borohydride (0.091 g, 2.39 mmol) was added. After five hours, water (20 ml) was added and the solution was extracted with chloroform (3×20 mL). The extracts were concentrated, and chromatographed (5% methanol in DCM) to yield the title compound as a white solid.

¹H NMR (DMSO-d₆): δ 8.37 (1H, s), 8.30 (1H, d, J=8.4 Hz), 7.87 (1H, s), 7.70 (1H, dd, J=8.5, 1.4 Hz), 6.7-6.9 (2H, br s), 4.4-4.5 (1H, br s), 4.40 (2H, s).

### EXAMPLE 14

### N-[(benzo[b]thien-3-yl)methyl]-sulfamoylpyrrolidine (Compound #101)

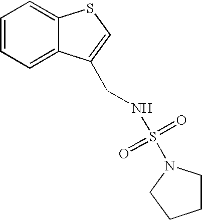

N-[(Benzo[b]thien-3-yl)methyl]-sulfamide (0.250 g, 1.03 mmol) and pyrrolidine (0.25 mL) were combined in anhydrous dioxane (5 mL) and heated to reflux for 32 hours. The reaction was evaporated and chromatographed with 5% methanol in DCM to yield the title compound as a white solid.

¹H NMR (CDCl₃): δ 7.84-7.89 (2H, m), 7.38-7.45 (3H, m), 4.49 (3H, br s), 3.25 (4H, t, J=4.0 Hz), 1.80 (4H, t, J=4.0 Hz).

### EXAMPLE 15

### N-[(benzo[b]thien-3-yl)methyl]-N′-ethylsulfamide (Compound #21)

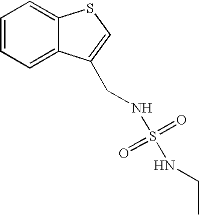

N-[(Benzo[b]thien-3-yl)methyl]-sulfamide (0.250 g, 1.03 mmol) and ethylamine (70% in H₂O, 0.10 mL) were combined in anhydrous dioxane (5 mL) and heated to reflux for 32 hours. The reaction was evaporated and chromatographed with 5% methanol in DCM to yield the title compound as a white solid.

¹H NMR (CDCl₃): δ 7.83-7.90 (2H, m), 7.36-7.47 (3H, m), 4.51 (2H, s), 2.90 (2H, q, J=7 Hz), 1.03 (3H, t, J=7 Hz).

### EXAMPLE 16

### Imidazole-1-sulfonic acid [(benzo[b]thien-3-yl)methyl]-amide (Compound #102)

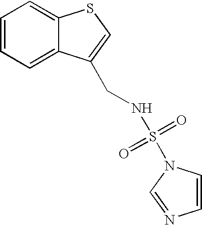

3-Benzothienylmethylamine and 3-(imidzole-1-sulfonyl)-1-methyl-3H-imidazol-1-ium triflate were combined in anhydrous acetonitrile. The solution was stirred at room temperature overnight, concentrated, and chromatographed (5% methanol in DCM) to yield the title compound as a tan solid.

¹H NMR (DMSO-d₆): δ 8.05 (1H, dd, J=7.0, 1.6 Hz), 7.99 (1H, dd, J=7.1, 1.7 Hz), 7.85 (1H, s), 7.66 (1H, s), 7.42-7.65 (5H, m), 4.34 (2H, s).

### EXAMPLE 17

### Dominant-Submissive In Vivo Assay

Dominance and submissiveness, defined in a competition test and measured as the relative success of two food-restricted rats to gain access to a feeder, form a behavioral paradigm—the Dominant Submissive Relationship (DSR). This paradigm results in two models which are predictive of ability of test compounds (drugs) to treat mood disorders.

Test compounds are evaluated for either their ability to inhibit the dominant behavior of rats taking food at the expense of an opponent (reduction of dominant behavior model or RDBM) and thus their potential as treatments for mania; or for their ability to increase the competitive behavior of submissive rats losing such encounters (reduction of submissive behavior model or RSBM) and thus their potential as treatments for depression; (Malatynska, E., and Knapp, R. J., Neuroscience and Biobehavioral Review, 29 (2005) 715-737).

The Reduction of Dominant Behavior Model (RDBM) test, wherein the dominant animals are treated with test compound, is predictive of the ability of the test compound to treat mani. This model was applied to Compound #1 of the present invention, according to the following procedure.

Male Sprague Dawley rats (140 to 160 g) from Charles River Laboratories Wilmington, Mass. were used in this assay. Shipments of rats were received at two-week intervals. Each shipment went through five-day quarantine, one-week acclimation period, and one-week selection process, followed by five-weeks of drug or vehicle treatment to those pairs selected.

Rats were housed four per cage. Access to food was restricted to one hour per day after testing on Monday through Thursday. After testing on Friday, rats had free access to food until being fasted again on Sunday. At no time were the rats deprived of water. The food deprivation periods used had little effect on weight gain as the average weight of rats was about 300 g at the end of the study. At the conclusion of experiment rats were sacrificed by decapitation, the trunk blood and brains were collected for in vitro experiments and drug concentration measurements.

The basic testing apparatus consisted of two chambers connected with a tunnel only large enough to allow one rat to pass through at a time. On the floor, at the mid-point of the tunnel was a container of sweetened milk. This basic apparatus was replicated, so that a total of four pairs of rats can be video tracked simultaneously. The camera can distinguish rats marked by different colors. Thus, the rats' heads were colored for the purpose of video tracking, red in one cage and yellow in the other cage. Only one animal at a time can have comfortable access to the feeder, but both animals can drink milk during the five-minute daily session. During the five-minute daily sessions, time spent in the feeder zone by each rat was recorded by the video tracking software and saved into a text file.

The test began with a random assignment of rats into pairs. Each member of a pair was placed in an opposite chamber of the testing apparatus. The time spent in the feeder zone by each animal was recorded. During the first week (five days) of testing the animals habituate to the new environment. Dominance was assigned to the animal with the highest score during the second week of testing if three criteria were achieved. First, there must have been a significant difference (two-tailed t-test, P<0.05) between the average daily drinking scores of both animals. Second, the dominant animal score must have been at least 25% greater than the submissive animal's score. Finally, there must have been no “reversals” during the pair selection week where the putative submissive rat out-scored its dominant partner on isolated occasions. Ideally there were minimal reversals during the acclimation week as well. About twenty-five to thirty-three percent of the initial animal pairs achieved these criteria and only these pairs were continued in the study.

Significant differences between time spent on the feeder by dominant and submissive rats were determined by ANOVA using GraphPad Prism software (GraphPad Software, Inc. San Diego, Calif.) followed by a two-tailed t-test (P<0.05). Comparisons were made between treatment groups using normalized dominance level values in paired animals. The dominance level is a value that measures social relation between paired subjects. Dominance level (DL)=FTD−FTS where FTD is the feeder time of dominant rats and FTS is the feeder time of submissive rats. The normalization was conducted according to the formula:

Dominance Level (week n in %)=(Dominance Level (week n))/(Dominance Level (week 2)

The statistical significance of the difference in dominance level between the control group (pairs of rats where both dominant and submissive animals were treated with vehicle) and the treatment group (submissive rats were treated with drug and dominant rats with vehicle) was determined by ANOVA, followed by a t-test. The normalized DL values were used for this calculation, where DL values for treatment weeks were normalized as a percent of the second week (pretreatment) value of that pair according the above formula. In these settings the minimum of the response (DL) determined drug positive activity, corresponding to efficacy, since DL values were always reduced if the response to a drug was positive. In the case of the negative response to a drug (worsening of symptoms) DL values were increased. If the drug did not have such activity the maximum of the response did not exceed 100%. Any maximal DL value significantly higher then control value (about 100%) indicated drug negative activity.

Compound #1 was evaluated in the Rat Reduction of Dominant Behavior Model, according to the procedure described in more detail below.

In Reduction of Dominance Behavior Model (RDBM), four groups of dominant rats were treated BID with Compound #1; one treated at 1.25 mg/kg (n=3), a second at 6 mg/kg (n=6), a third at 30 mg/kg (n=6) and a fourth at 60 mg/kg dose (n=7). A fifth group of dominant rats was treated with lithium chloride at 100 mg/kg/QD (n=6), and a sixth with 0.5% methylcellulose (vehicle control). All treatments started on Saturday after the second testing week (selection week). Fluoxetine and lithium chloride were injected intraperitoneally (i.p.) once a day (QD). Compound #1 was administered orally (p.o) twice a day (BID). Approximate time of the first daily dose was between 7:00 a.m. and 8:00 a.m. and the second daily dose between 4:00 p.m. and 6:00 p.m.

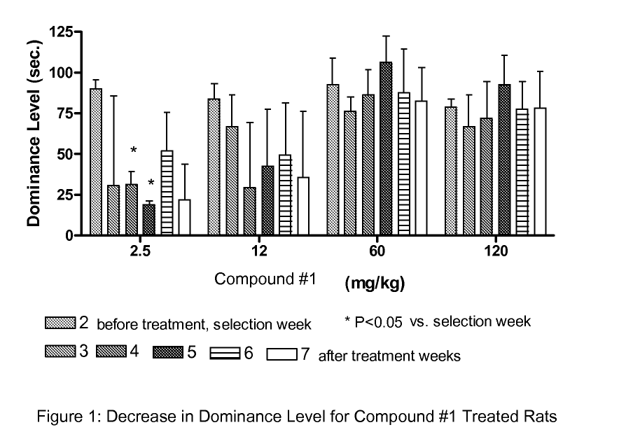

FIG. 1 illustrates the measured decrease in dominance level for rats treated with Compound #1 as a function of weeks in the study. Compound #1 reduced dominant behavior in a dose-dependent manner indicating that the compound is active as an anti-manic agent at low doses. The reduction of dominant behavior was statistically significant after 2 weeks of treatment with 1.25 mg/kg (p<0.05).

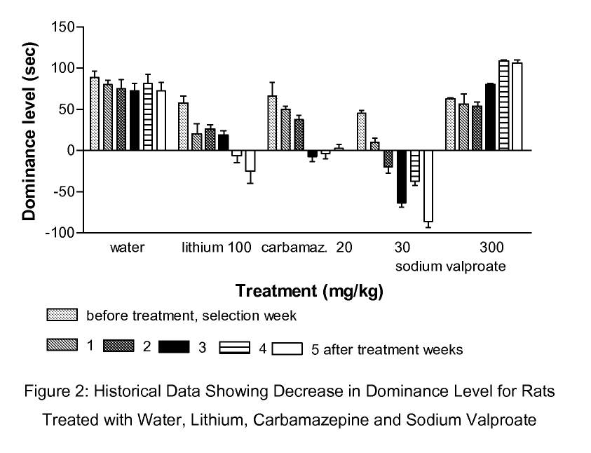

FIG. 2 illustrates historical data on the measured decrease in dominance level for rats treated with water, lithium (100 mg/kg, i.p.), carbamazepine (20 mg/kg, i.p.) and sodium valproate (30 and 300 mg/klg, i.p.). Dominant behavior was significantly reduced by lithium after 1 week, carbamazepine after 3 weeks and 30 mg/kg VPA only after 1 week of treatment.

### EXAMPLE 18

### Dominant-Submissive In Vivo Assay

The Reduction of Submissive Behavior Model (RSBM) test, wherein the submissive animals are treated with test compound, is predictive of the ability of the test compound to treat depression. This model was applied to Compound #1 of the present invention, according to the following procedure.

Male Sprague Dawley rats (140 to 160 g) from Charles River Laboratories Wilmington, Mass. were used in this assay. Shipments of rats were received at two-week intervals. Each shipment went through five-day quarantine, one-week acclimation period, and one-week selection process, followed by five-weeks of drug or vehicle treatment to those pairs selected.

Rats were housed four per cage. Access to food was restricted to one hour per day after testing on Monday through Thursday. After testing on Friday, rats had free access to food until being fasted again on Sunday. At no time were the rats deprived of water. The food deprivation periods used had little effect on weight gain as the average weight of rats was about 300 g at the end of the study. At the conclusion of experiment rats were sacrificed by decapitation, the trunk blood and brains were collected for in vitro experiments and drug concentration measurements.

The basic testing apparatus consisted of two chambers connected with a tunnel only large enough to allow one rat to pass through at a time. On the floor, at the mid-point of the tunnel was a container of sweetened milk. This basic apparatus was replicated, so that a total of four pairs of rats can be video tracked simultaneously. The camera can distinguish rats marked by different colors. Thus, the rats' heads were colored for the purpose of video tracking, red in one cage and yellow in the other cage. Only one animal at a time can have comfortable access to the feeder, but both animals can drink milk during the five-minute daily session. During the five-minute daily sessions, time spent in the feeder zone by each rat was recorded by the video tracking software and saved into a text file.

The test began with a random assignment of rats into pairs. Each member of a pair was placed in an opposite chamber of the testing apparatus. The time spent in the feeder zone by each animal was recorded. During the first week (five days) of testing the animals habituate to the new environment. Dominance was assigned to the animal with the highest score during the second week of testing if three criteria were achieved. First, there must have been a significant difference (two-tailed t-test, P<0.05) between the average daily drinking scores of both animals. Second, the dominant animal score must have been at least 25% greater than the submissive animal's score. Finally, there must have been no “reversals” during the pair selection week where the putative submissive rat out-scored its dominant partner on isolated occasions. Ideally there were minimal reversals during the acclimation week as well. About twenty-five to thirty-three percent of the initial animal pairs achieved these criteria and only these pairs were continued in the study.

Significant differences between time spent on the feeder by dominant and submissive rats were determined by ANOVA using GraphPad Prism software (GraphPad Software, Inc. San Diego, Calif.) followed by a two-tailed t-test (P<0.05). Comparisons were made between treatment groups using normalized dominance level values in paired animals. The dominance level is a value that measures social relation between paired subjects. Dominance level (DL)=FTD−FTS where FTD is the feeder time of dominant rats and FTS is the feeder time of submissive rats. The normalization was conducted according to the formula:

Dominance Level (week n in %)=(Dominance Level (week n))/(Dominance Level (week 2)

The statistical significance of the difference in dominance level between the control group (pairs of rats where both dominant and submissive animals were treated with vehicle) and the treatment group (submissive rats were treated with drug and dominant rats with vehicle) was determined by ANOVA, followed by a t-test.

The activity onset time value at 50% of response (AOT-50) and the minimum and maximum response to drug were calculated based on the reduction of the dominance level value using non-linear regression analysis (GraphPad Software, Inc., San Diego, Calif.). The normalized DL values were used for this calculation, where DL values for treatment weeks were normalized as a percent of the second week (pretreatment) value of that pair according the above formula. In these settings the minimum of the response (DL) determined drug positive activity, corresponding to efficacy, since DL values were always reduced if the response to a drug was positive. In the case of the negative response to a drug (worsening of symptoms) DL values were increased. If the drug did not have such activity the maximum of the response did not exceed 100%. Any maximal DL value significantly higher then control value (about 100%) indicated drug negative activity.

Compound #1 was evaluated in the Rat Reduction of Submissive Behavior Model (RSBM) of depression (this is a mice paper, wrong journal) (Malatynska E, Goldenberg R, Shuck L, Haque A, Crites G, and Knapp R. Reduction of submissive behavior as a model of depression. Pharmacol. 1:1-9 2002; Malatynska, E., and Knapp, R. J., Neuroscience and Biobehavioral Review, 29 (2005) 715-737); Pinhasov, A., Crooke, J., Rosenthal, D., Brenneman D. E., and Malatynska, E. Reduction of Submissive Behavior Model for antidepressant drug activity testing: study using a video-tracking system. Behav Pharmacol. December; 16(8):657-64, 2005.

Submissive rats were dosed orally with vehicle (0.5% aqueous solution of methylcellulose) or Compound #1 (6, 30, and 60 mg/kg; n=6 rats/group) twice a day for 5 weeks while their dominant partners were treated with vehicle. Compound #1 reduced submissive behavior (antidepressant) in a dose-dependent manner. The reduction of submissive behavior was statistically significant after 5 weeks of treatment with 30 mg/kg (p<0.05) and after 17 days with 60 mg/kg dose (p<0.01). A decrease in the submissive response indicates an increase in dominance level and is predictive of antidepressant activity.

### EXAMPLE 19-21

### Kindling and Bipolar Cycling

Discussion in current literature suggests that the mechanisms underlying kindling may be similar to the mechanism of cycling in bipolar disorder and/or may be related to mood stabilization. Thus, the amygdala kindling and hippocampal kindling assays described in more detail hereinafter may be predictive of the ability of a test compound to treat the cycling associated with, characteristic of or symptomatic of bipolar disorder. (Ghaemi, S. N., Boiman, E. E., and Goodwin, F. K., Soc. of Bio. Psychiatry, (1999), vol. 45, pp 137-144; Stoll, A. L., and Severus, W. E., Harvard Rev. Psychiatry, July/August (1996), Vol. 4, No. 2, pp 77-89)

### EXAMPLE 19

### Amygdala Kindling Assay (Kindling Prevention)

Briefly, the assay procedure was as follows. Adult male, Sprague-Dawley rats weighing between 250-300 g were obtained from Charles River, Wilmington, Mass. All the animals were housed on a 12:12 light dark cycle and permitted free access to both food (Prolab RMH 3000) and water except when removed from the home cage for experimental procedures. Animals were cared for in a matter consistent with the recommendations detailed in the National Research Council Publication, “Guide for the Care and Use of Laboratory Animals” in a temperature controlled, pesticide-free facility. Kindling stimulations were routinely performed between 9 AM-2 PM to avoid any circadian variations.

The ability of Compound #1 to block the expression of amygdala kindled seizures was determined as follows. Rats were anaesthetized with a ketamine (120 mg/kg, i.p.) and xylazine (12 mg/kg, i.p.) cocktail. Under aseptic conditions, a bipolar electrode (Plastic One, Roanoke, Va.) was stereotaxically implanted into the right basolateral amygdala (AP-2.2, ML-4.7, DV-8.7; Paxinos and Watson). Anterior-posterior and lateral measurements were from Bregma, whereas the dorsal-ventral measurement was from the skull surface. Sterile skull screws (3-4) were implanted for the indifferent reference electrode. Electrodes were fixed using dental cement and acrylic. The wound was then closed using sterile 18/8 Michel suture clips (Roboz, Gaithersburg, Md.). Neomycin antibiotic ointment was applied to the wound and a single dose of penicillin (60,000 IU, im, AgriLabs) was administered to the each rat before returning them to clean cages for one week of post operative recovery.

Amygdala kindling was then performed according to the following protocol. Following a brief acclimation (<5 minutes) to the recording chamber, baseline EEG recordings were obtained (MP 100, Biopac Systems Inc., Goleta, Calif.). Rats were then randomized to receive either vehicle (0.5% methylcellulose) or Compound #1 (100 mg/kg, i.p.) (n=10 rats per group). On the day of the experiment, a single dose of Compound #1 or 0.5% methylcellulose was administered 30 minutes prior to amygdala stimulation (200 μA for 2 seconds). The behavioral seizure score and AD duration was recorded for rats in each treatment group. Behavioral seizure scores were determined using the Racine scale; i.e., 0=no response; stage 1=grooming/hyperactivity; stage 2=head nodding/tremor; stage 3=unilateral forelimb clonus; stage 4=clonus with rearing; and stage 5=generalized tonic-clonic seizure with rearing and falling (Racine, 1972). After-discharge (AD) activity was digitally recorded for up to 180 seconds following the stimulation train and the duration of the primary AD was measured. Rats were considered fully kindled when they displayed five consecutive Stage 4 or 5 generalized seizures. Daily stimulations were continued for up to 13 consecutive days in all three groups until rats in the vehicle-treated group were fully kindled (i.e., five consecutive Stage 4 or 5 seizures). At this time, all rats were allowed a one-week stimulus and drug-free period; after which they were re-challenged in the absence of drug with the same stimulus employed during the acquisition phase (i.e., days 1-13). Rats treated with Compound #1 were subsequently stimulated once per day until they reached a fully kindled state.

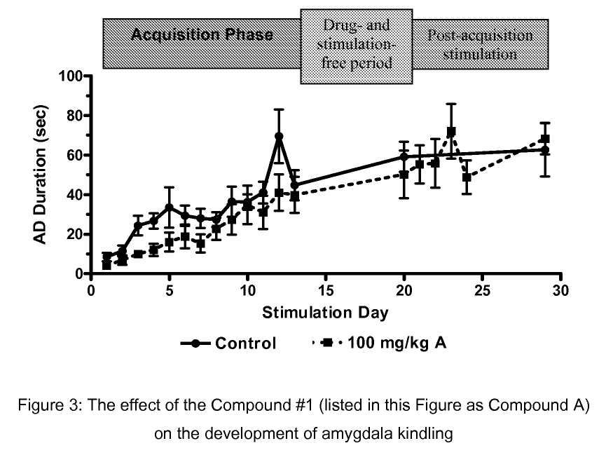

The after discharge (AD) duration in the vehicle and Compound #1 treatment groups displayed a progressive increase over the course of the kindling acquisition phase, as shown in FIG. 3. No statistical difference between treatment groups was observed.

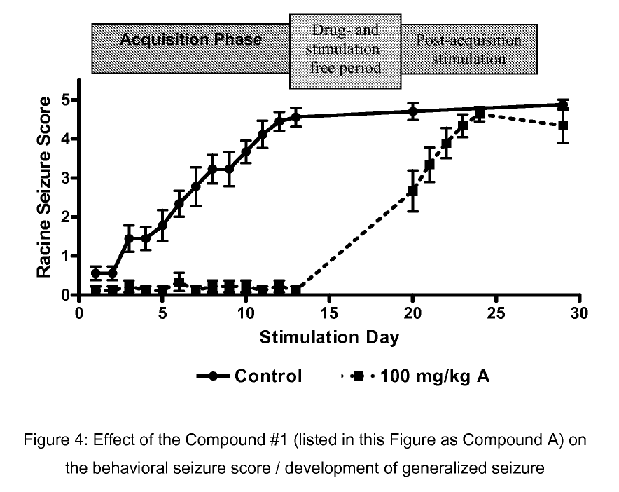

Compound #1 prevented the acquisition of the fully generalized kindled seizure as shown in FIG. 4. This conclusion is based on the finding that the seizure score at the conclusion of the drug- and stimulus-free period remained significantly lower than that of the rats in the vehicle-treated group. Furthermore, when stimulated in the absence of drug, the seizure score of rats treated with Compound #1 increased at a rate that was parallel to that observed in the vehicle-treated rats.

The results demonstrate that Compound #1 possesses the ability to modify the development of kindling in the amygdala kindled rat model of partial epilepsy. These results are consistent with the conclusion that Compound #1 possesses disease-modifying effects. This conclusion is based on the finding that the seizure score, at the conclusion of the drug- and stimulation-free period, of rats treated with Compound #1 remained significantly lower than that of the vehicle-treated rats. Furthermore, once the stimulation protocol was resumed in the absence of drug, the seizure score progressed at a rate that was parallel to the vehicle-treated group.

The kindled rat model of partial epilepsy is an accepted model of epileptogenesis (McNamara, 1995). The finding that the seizure score, but not the after-discharge duration, in both treatment groups one-week after the stimulus- and drug-free week was markedly lower than that of the vehicle-treated groups suggest that both drugs prevent the acquisition of the secondarily generalized seizure but not the focal seizure. The results obtained with Compound #1 in the present study are similar in magnitude to those obtained with the antiepileptic drugs valproic acid (Silver et al., 1991), phenobarbital (Silver et al., 1991), levetiracetam (Loscher et al., 1998), topiramate (Amano et al., 1998) and the NMDA receptor antagonist MK-801 (Morimoto et al., 1991). The present results suggest that Compound #1 may prevent the development of epilepsy in susceptible patients.

### EXAMPLE 20

### Rat Hippocampal Kindling Model (Interruption of Kindled State)

Kindled seizures provide an experimental model of focal seizures, allowing scientists to study complex brain networks that may contribute to seizure spread and generalization from a focus.

In the present rapid hippocampal kindling model adult male Sprague-Dawley rats (300-400 g) were surgically implanted with bipolar electrodes placed in the hippocampus. Rats were kindled by repetitive electrical stimulation (50 Hz, 10 s train of 1 ms, biphasic 200 μA pulses every 30 min for 6 h every other day for a total of 60 stimulations) resulting in stage 5 bilateral motor seizures. One week later, the rats received 2-3 supra-threshold stimulations delivered every 30 min before compound treatment to ensure stability of the behavioral seizure stage and after-discharge duration. Fifteen minutes after the last stimulation, a single dose of vehicle or test compound was administered i.p (intraperitoneally). After 15 min, each rat was then stimulated every 30 min for 3 to 4 h. After each stimulation, individual seizure scores and after-discharge durations were recorded. The group mean±SEM were calculated for each parameter. Eight rats per dose and a minimum of four doses were used to establish an ED₅₀ value. Efficacy was measured as the ability of a compound to modify the seizure score (severity of spread) and after-discharge duration (ADD; excitability) of the generalized seizures.

Using this approach, a compound that reduces the seizure score from 5 to 3 without any effect on the ADD suggest the utility of the compound for the treatment of secondarily generalized seizures. In contrast, a compound that reduces the seizure score from 5 to less than 1, as well as reduces the ADD, suggest the utility of the compound for the treatment of focal seizures. Thus, according to the theories presented in current literature (Ghaemi, S. N., Boiman, E. E., and Goodwin, F. K., Soc. of Bio. Psychiatry, (1999), vol. 45, pp 137-144; Stoll, A. L., and Severus, W. E., Harvard Rev. Psychiatry, July/August (1996), Vol. 4, No. 2, pp 77-89) a decrease in seizure score and/or ADD may also be predictive of the ability of a test compound to treat the cycling associated with bipolar disorder.

Compound #1 (formulated in a 0.5% aqueous solution of methylcellulose) exhibited anticonvulsant activity in this model with an ED₅₀=38.9±0.9 mg/kg (peak activity at 15 min; sustained at 1 h). Seizure scores were significantly reduced from 5 to 0 in 6 out of 8 rats and the ADD also decreased by a statistically significant amount (75%; p<0.01). Ethosuximide was ineffective in this model; whereas, phenytoin, carbamazepine and valproic acid suppressed seizure activity, but at doses associated with toxicity.

In this model, 6 out of 8 rats showed complete absence of seizure activity after treatment with Compound #1. Similar protection was observed with valproic acid, but only at toxic doses (>300 mg/kg). Comparison Results from this assay are as listed in Table 4, below.

TABLE 4
Evaluation of JNJ-26489112 and Reference Drugs in the Rat
Hippocampal Kindling Test

| | | | After-Discharge |
|---|---|---|---|
| | | Mean Seizure | Duration |
| | Dose (mg/kg), i.p. | Score | (% of control) |
| Compound #1 | ED₅₀ = 38.9 | 1.6 ± 0.7 | 25 ± 9% |
| | TD₅₀ = 100 | 0.9 ± 0.6 | |
| Ethosuximide | ED₅₀ = 250 | 5 ± 0 | 78 ± 13% |
| | TD₅₀ = 189 | | |
| Phenytoin | ED₅₀ = 150 | 4.3 ± 0.3 | 209 ± 43% |
| | TD₅₀ = 15 | | |
| Carbamazepine | ED₅₀ = 50 | 2.3 ± 0.6 | 72 ± 13% |
| | TD₅₀ = 26 | | |
| Valproic acid | ED₅₀ = 350 | 0.3 ± 0.3 | 3 ± 1.4% |
| | TD₅₀ = 316 | | |

ED₅₀ = median therapeutic dose;
TD₅₀ = median toxic dose
P = paired t-test; one-tailed

### EXAMPLE 21

### Lamotrigine Resistant Amygdala Kindled Rat Model (Interruption of Kindled State)

Compound #1 was evaluated in the lamotrigine (LTG)-resistant amygdala kindled rat model. Amygdala kindling is less severe than hippocampal kindling, such that many AEDs are effective against amygdala kindled seizures, but are ineffective against hippocampal kindled seizures. For example, LTG can significantly reduce amygdala kindled seizure score and ADD (ED₅₀=25 mg/kg, i.p., Cl=4-50 mg/kg; score ˜2; ADD reduced by 62%), but is unable to protect against hippocampal kindled seizures.

In the LTG-resistant amygdala kindling model, rats were dosed with LTG (5 mg/kg, i.p., q.d.) during the acquisition phase of kindling. This dose has been shown to have no effect on kindling itself, but leads to the development of fully kindled rats that are resistant to the anticonvulsant effects of LTG. Once kindled (supra-threshold stimulation of 150 μA biphasic 60 Hz current pulse for 1 second; ˜2 weeks), rats were re-challenged with a high dose of LTG (45 mg/kg, i.p.) one week later to insure resistance. After a 3-4 day wash out period, rats received 2-3 supra-threshold stimulations delivered every 30 min before compound treatment to ensure stability of the behavioral seizure stage and after-discharge duration. Fifteen minutes after the last stimulation, a single dose of vehicle or test compound was administered i.p. After 15 min, each rat was then stimulated every 30 min for 3 to 4 h. After each stimulation, individual seizure scores and after-discharge durations were recorded. The group mean±SEM was calculated for each parameter.

Compound #1 (100 mg/kg, i.p., n=11) significantly reduced the seizure score and after-discharge duration. Five out of eleven rats were protected such that the seizure score was reduced from a 5 to 3.1 and the after-discharge duration was reduced 72% (from 112 sec to 32 sec).

### EXAMPLE 22

As a specific embodiment of an oral composition, 100 mg of the Compound #1 prepared as in Example 1 is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A method of treating mania comprising administering to a subject in need thereof a therapeutically effective amount of a compound of the formula (I) 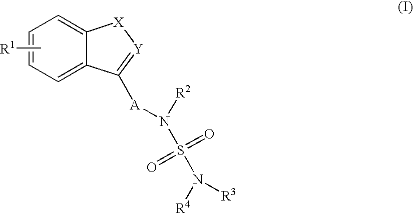
wherein
R¹ is selected from the group consisting of hydrogen, halogen, hydroxy, methoxy, trifluoromethyl, nitro and cyano;
X—Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen and C₁₋₄alkyl;
alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

2. The method of claim 1 wherein
R¹ is selected from the group consisting of hydrogen, halogen, trifluoromethyl, cyano and nitro;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen, methyl and ethyl;
or a pharmaceutically acceptable salt thereof.

3. The method of claim 2, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each independently selected from the group consisting of hydrogen and ethyl;
or a pharmaceutically acceptable salt thereof.

4. The method of claim 3, wherein
R¹ is selected from the group consisting of hydrogen, 5-chloro, 5-fluoro, 5-bromo, 4-bromo, 7-fluoro, 5-trifluoromethyl and 5-cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each hydrogen; alternatively R³ is hydrogen and R⁴ is ethyl;
or a pharmaceutically acceptable salt thereof.

5. The method of claim 1, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

6. The method of claim 5, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 6 membered, saturated or aromatic ring structure, optionally containing one to two additional heteroatoms independently selected from the group consisting of O, N and S;
or a pharmaceutically acceptable salt thereof.

7. The method of claim 6, wherein
R¹ is hydrogen;
X—Y is —S—CH—;
A is —CH₂—;
R² is hydrogen;
R³ and R⁴ are taken together with the nitrogen atom to which they are bound to form a 5 membered ring structure selected from the group consisting of pyrrolidinyl and imidazolyl;
or a pharmaceutically acceptable salt thereof.

8. The method of claim 2, wherein the compound of formula (I) is selected from the group consisting of
N-(benzo[b]thien-3-ylmethyl)-sulfamide;
N-[(5-chlorobenzo[b]thien-3-yl)methyl]-sulfamide;
N-(3-benzofuranylmethyl)-sulfamide;
N-[(5-fluorobenzo[b]thien-3-yl)methyl]-sulfamide;
N-(1-benzo[b]thien-3-ylethyl)-sulfamide;
N-(1-naphthalenylmethyl)-sulfamide;
N-[(2-methyl-3-benzofuranyl)methyl]-sulfamide;
N-[(5-bromobenzo[b]thien-3-yl)methyl]-sulfamide;
N-[(4-bromobenzo[b]thien-3-yl)methyl]-sulfamide;
N-[(7-fluorobenzo[b]thien-3-yl)methyl]-sulfamide;
N-[(1-methyl-1H-indol-3-yl)methyl]-sulfamide;
N-[(4-trifluoromethylbenzo[b]thien-3-yl)methyl]-sulfamide;
N-[(4-cyanobenzo[b]thien-3-yl)methyl]-sulfamide;
N-[(benzo[b]thien-3-yl)methyl]-sulfamoylpyrrolidine;
N-[(benzo[b]thien-3-yl)methyl]-N′-ethylsulfamide;
imidazole-1-sulfonic acid [(benzo[b]thien-3-yl)methyl]-amide;
and pharmaceutically acceptable salts thereof.

9. The method of claim 1, wherein the compound of formula (I) is selected from the group consisting of N-(benzo[b]thien-3-ylmethyl)-sulfamide; N-[(5-fluorobenzo[b]thien-3-yl)methyl]-sulfamide; and pharmaceutically acceptable salts thereof.

10. A method of treating mania comprising administering to a subject in need thereof a therapeutically effective amount of a compound selected from the group consisting of N-(benzo[b]thien-3-ylmethyl)-sulfamide and pharmaceutically acceptable salts thereof.

11. A method of treating bipolar disorder comprising administering to a subject in need thereof a therapeutically effective amount of a compound of the formula (I) 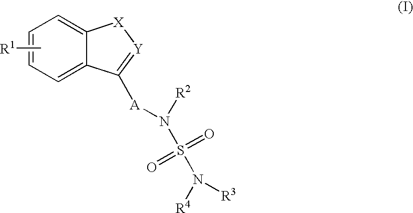
wherein
R¹ is selected from the group consisting of hydrogen, halogen, hydroxy, methoxy, trifluoromethyl, nitro and cyano;
X—Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen and C₁₋₄alkyl;
alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

12. The method of claim 11, wherein
R¹ is selected from the group consisting of hydrogen, halogen, trifluoromethyl, cyano and nitro;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen, methyl and ethyl;
or a pharmaceutically acceptable salt thereof.

13. The method of claim 12, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each independently selected from the group consisting of hydrogen and ethyl;
or a pharmaceutically acceptable salt thereof.

14. The method of claim 13, wherein
R¹ is selected from the group consisting of hydrogen, 5-chloro, 5-fluoro, 5-bromo, 4-bromo, 7-fluoro, 5-trifluoromethyl and 5-cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each hydrogen; alternatively R³ is hydrogen and R⁴ is ethyl;
or a pharmaceutically acceptable salt thereof.

15. The method of claim 11, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

16. The method of claim 15, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 6 membered, saturated or aromatic ring structure, optionally containing one to two additional heteroatoms independently selected from the group consisting of O, N and S;
or a pharmaceutically acceptable salt thereof.

17. The method of claim 16, wherein
R¹ is hydrogen;
X—Y is —S—CH—;
A is —CH₂—;
R² is hydrogen;
R³ and R⁴ are taken together with the nitrogen atom to which they are bound to form a 5 membered ring structure selected from the group consisting of pyrrolidinyl and imidazolyl;
or a pharmaceutically acceptable salt thereof.

18. The method of claim 12, wherein the compound of formula (I) is selected from the group consisting of
N-(benzo[b]thien-3-yl methyl)-sulfamide;
N-[(5-chlorobenzo[b]thien-3-yl)methyl]-sulfamide;
N-(3-benzofuranylmethyl)-sulfamide;
N-[(5-fluorobenzo[b]thien-3-yl)methyl]-sulfamide;
N-(1-benzo[b]thien-3-ylethyl)-sulfamide;
N-(1-naphthalenylmethyl)-sulfamide;
N-[(2-methyl-3-benzofuranyl)methyl]-sulfamide;
N-[(5-bromobenzo[b]thien-3-yl)methyl]-sulfamide;
N-[(4-bromobenzo[b]thien-3-yl)methyl]-sulfamide;
N-[(7-fluorobenzo[b]thien-3-yl)methyl]-sulfamide;
N-[(1-methyl-1H-indol-3-yl)methyl]-sulfamide;
N-[(4-trifluoromethylbenzo[b]thien-3-yl)methyl]-sulfamide;
N-[(4-cyanobenzo[b]thien-3-yl)methyl]-sulfamide;
N-[(benzo[b]thien-3-yl)methyl]-sulfamoylpyrrolidine;
N-[(benzo[b]thien-3-yl)methyl]-N′-ethylsulfamide;
imidazole-1-sulfonic acid [(benzo[b]thien-3-yl)methyl]-amide;
and pharmaceutically acceptable salts thereof.

19. The method of claim 11, wherein the compound of formula (I) is selected from the group consisting of N-(benzo[b]thien-3-ylmethyl)-sulfamide; N-[(5-fluorobenzo[b]thien-3-yl)methyl]-sulfamide; and pharmaceutically acceptable salts thereof.

20. A method of treating mania comprising administering to a subject in need thereof a therapeutically effective amount of a compound selected from the group consisting of N-(benzo[b]thien-3-ylmethyl)-sulfamide and pharmaceutically acceptable salts thereof.

21. The method of claim 11, wherein the method of treating bipolar disorder comprises treating the depression and the mania of bipolar disorder.

22. The method of claim 11, wherein the method of treating bipolar disorder comprises treating the depression, the mania and the cycling of bipolar disorder.

23. The method of claim 20, wherein the method of treating bipolar disorder comprises treating the depression and the mania of bipolar disorder.

24. The method of claim 20, wherein the method of treating bipolar disorder comprises treating the depression, the mania and the cycling bipolar disorder.

25. A method of treating bipolar depression comprising administering to a subject in need thereof a therapeutically effective amount of a compound of the formula (I) 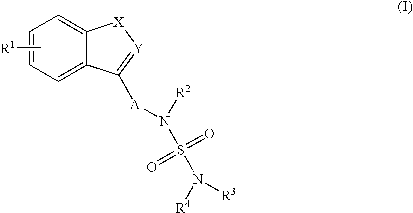
wherein
R¹ is selected from the group consisting of hydrogen, halogen, hydroxy, methoxy, trifluoromethyl, nitro and cyano;
X—Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen and C₁₋₄alkyl;
alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

26. The method of claim 25, wherein
R¹ is selected from the group consisting of hydrogen, halogen, trifluoromethyl, cyano and nitro;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen, methyl and ethyl;
or a pharmaceutically acceptable salt thereof.

27. The method of claim 26, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R²is hydrogen;
R³ and R⁴ are each independently selected from the group consisting of hydrogen and ethyl;
or a pharmaceutically acceptable salt thereof.

28. The method of claim 27, wherein
R¹ is selected from the group consisting of hydrogen, 5-chloro, 5-fluoro, 5-bromo, 4-bromo, 7-fluoro, 5-trifluoromethyl and 5-cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each hydrogen; alternatively R³ is hydrogen and R⁴ is ethyl;
or a pharmaceutically acceptable salt thereof.

29. The method of claim 25, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

30. The method of claim 29, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 6 membered, saturated or aromatic ring structure, optionally containing one to two additional heteroatoms independently selected from the group consisting of O, N and S;
or a pharmaceutically acceptable salt thereof.

31. The method of claim 30, wherein
R¹ is hydrogen;
X—Y is —S—CH—;
A is —CH₂—;
R² is hydrogen;
R³ and R⁴ are taken together with the nitrogen atom to which they are bound to form a 5 membered ring structure selected from the group consisting of pyrrolidinyl and imidazolyl;
or a pharmaceutically acceptable salt thereof.

32. The method of claim 26, wherein the compound of formula (I) is selected from the group consisting of
N-(benzo[b]thien-3-yl methyl)-sulfamide;
N-[(5-chlorobenzo[b]thien-3-yl)methyl]-sulfamide;
N-(3-benzofuranylmethyl)-sulfamide;
N-[(5-fluorobenzo[b]thien-3-yl)methyl]-sulfamide;
N-(1-benzo[b]thien-3-ylethyl)-sulfamide;
N-(1-naphthalenylmethyl)-sulfamide;
N-[(2-methyl-3-benzofuranyl)methyl]-sulfamide;
N-[(5-bromobenzo[b]thien-3-yl)methyl]-sulfamide;
N-[(4-bromobenzo[b]thien-3-yl)methyl]-sulfamide;
N-[(7-fluorobenzo[b]thien-3-yl)methyl]-sulfamide;
N-[(1-methyl-1H-indol-3-yl)methyl]-sulfamide;
N-[(4-trifluoromethylbenzo[b]thien-3-yl)methyl]-sulfamide;
N-[(4-cyanobenzo[b]thien-3-yl)methyl]-sulfamide;
N-[(benzo[b]thien-3-yl)methyl]-sulfamoylpyrrolidine;
N-[(benzo[b]thien-3-yl)methyl]-N′-ethylsulfamide;
imidazole-1-sulfonic acid [(benzo[b]thien-3-yl)methyl]-amide;
and pharmaceutically acceptable salts thereof.

33. The method of claim 25, wherein the compound of formula (I) is selected from the group consisting of N-(benzo[b]thien-3-ylmethyl)-sulfamide; N-[(5-fluorobenzo[b]thien-3-yl)methyl]-sulfamide; and pharmaceutically acceptable salts thereof.

34. A method of treating mania comprising administering to a subject in need thereof a therapeutically effective amount of a compound selected from the group consisting of N-(benzo[b]thien-3-ylmethyl)-sulfamide and pharmaceutically acceptable salts thereof.
